(19) United States
(12) Patent Application Publication
(10) Pub. No.: US 2024/0161881 A1
(43) Pub. Date: May 16, 2024

(54) COMMUNITY MANAGEMENT FOR A COMPLIANCE AND PREVENTION SYSTEM

(71) Applicant: NUCLEUS HEALTHCARE, LLC., Radnor, PA (US)

(72) Inventors: Steven PAOLUCCI, Radnor, PA (US); Brian O'NEIL, Radnor, PA (US)

(73) Assignee: NUCLEUS HEALTHCARE, LLC., Radnor, PA (US)

(21) Appl. No.: 18/550,250

(22) PCT Filed: Mar. 14, 2022
(86) PCT No.: PCT/US2022/020277

§ 371 Date: Sep. 12, 2023

Related U.S. Application Data
(60) Provisional application No. 63/160,643, filed on Mar. 12, 2021.

(51) Int. Cl. G16H 10/40; G06K 7/14

(52) U.S. Cl. CPC G16H 10/40; G06K 7/1413; G06K 7/1417

(57) ABSTRACT

A computer program product is provided. The computer program product includes processor executable code for an integration tracking engine. The processor executable code is stored on a non-transitory computer readable medium. The processor executable code is executed by at least one processor to cause the integration tracking engine to detect and scan a bar or quick response (QR) code corresponding to a vial, determine whether o samples corresponding to users are pooled into the vial, associating a pooling order with respect to the vial when the samples are pooled, determines if test results are positive for the vial; and associated a presumptively positive test result with each of the users when test results are positive.

### CROSS REFERENCE TO RELATED APPLICATION

This application claims the benefit of U.S. Provisional Application Ser. No. 63/160,643 filed on Mar. 12, 2021, which is incorporated by reference as if fully set forth.

### BACKGROUND

Ensuring a population well-being is a priority. Yet, conventional mechanisms for addressing new or emerging health crises have been found wanting, especially regarding safety related to preventing infection and spread of viruses, bacteria, and diseases during a pandemic. For instance, conventional mechanisms are deficient in tracking and reporting infection tests, ensuring timely notifications, and providing protocols to keep all employees safe. Currently, there is a need for a compliance and prevention system and architecture that can address new or emerging health crises.

### SUMMARY

According to one or more embodiments, a computer program product is provided. The computer program product includes processor executable code for an integration tracking engine. The processor executable code is stored on a non-transitory computer readable medium. The processor executable code is executed by at least one processor to cause the integration tracking engine to detect and scan a bar or quick response (QR) code corresponding to a user profile, load an order for the user profile, generate and present at least one user interface instructing a collecting of a sample within a vial of a test kit and scanning of a bar or QR code of the vial once filled with the sample, and associate the vial with the order, the user profile, and the vial.

According to one or more embodiments, a computer program product is provided. The computer program product includes processor executable code for an integration tracking engine. The processor executable code is stored on a non-transitory computer readable medium. The processor executable code is executed by at least one processor to cause the integration tracking engine to determine whether a user had a positive test with respect to an order, provide one or more notifications to the user indicating that a test was positive when the integration tracking engine determines that the user had the positive test with respect to the order, send an alert to a back-end system to cause a contact tracing operation with respect to the user, and trigger a perimeter management flagging the user as unable to enter one or more facilities.

According to one or more embodiments, a computer program product is provided. The computer program product includes processor executable code for an integration tracking engine. The processor executable code is stored on a non-transitory computer readable medium. The processor executable code is executed by at least one processor to cause the integration tracking engine to detect and scan a bar or quick response (QR) code corresponding to a vial, determine whether o samples corresponding to users are pooled into the vial, associating a pooling order with respect to the vial when the samples are pooled, determines if test results are positive for the vial; and associated a presumptively positive test result with each of the users when test results are positive.

According to one or more embodiments, the computer program product embodiments herein can be implemented as or in an apparatus, a system, and/or a method.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 depicts a compliance and prevention environment according to one or more embodiments;

FIG. 2 depicts a compliance and prevention system according to one or more embodiments;

FIG. 3 depicts a system, a neural network, and a method according to one or more embodiments;

FIG. 4 depicts a method according to one or more embodiments;

FIG. 5 depicts a method according to one or more embodiments;

FIG. 6 depicts a method according to one or more embodiments;

FIG. 7 depicts a method according to one or more embodiments;

FIG. 8 depicts a method according to one or more embodiments; and

FIG. 9 depicts a method according to one or more embodiments.

### DETAILED DESCRIPTION

Disclosed herein is an integration tracking engine executing across a compliance and prevention system and architecture.

The compliance and prevention system and architecture relates to an end-to-end solution that employs mechanisms and algorithms to provide patients, customers, companies, and governments medical data tracking, cross-platform digital management services, and disparate environment integration. According to one or more embodiments, the compliance and prevention system and architecture (by implementing the integration tracking engine) can include/provide test locations, test scheduling, sample tracking, daily symptom tracking, contact tracing assistance, dashboard compliance reporting, government reporting, and test kit activation. The integration tracking engine can be a processor executable code or software that is necessarily rooted in process operations by, and in processing hardware of, the compliance and prevention system and architecture.

One or more technical effects, advantages, or benefits of the compliance and prevention system and architecture include providing capabilities that can address new or emerging health crises. Further, the integration tracking engine and the compliance and prevention system and architecture can include providing a robust data intake for patients at testing sites that can be further integrated and propagated across disparate environments to eliminate lost sample, expired samples, lost data, delayed data and/or samples, data entry errors, chain of custody problems, and the like. Thus, the compliance and prevention system and architecture particularly utilizes the integration tracking engine, and the integration tracking engine transforms the compliance and prevention system and architecture to enable/implement these advantages, technical effects, and benefits (as well as others discussed herein) that are otherwise not currently available with conventional mechanisms or currently performed in market.

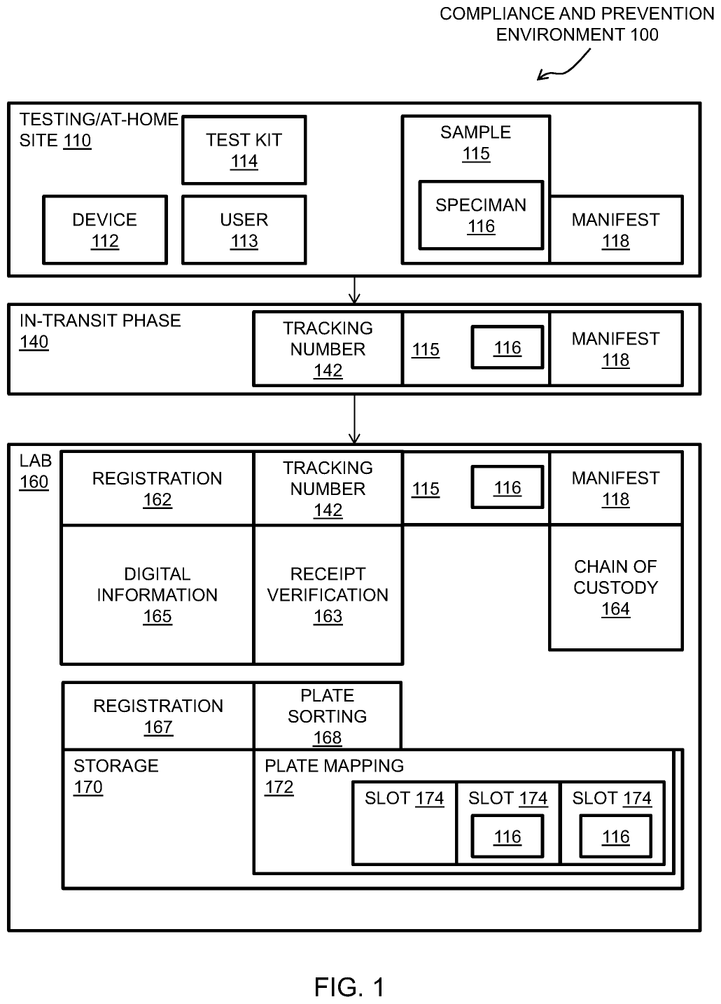

FIG. 1 depicts an environment 100 (e.g., an example of the compliance and prevention system) according to one or more embodiments. The environment 100 includes a testing/at-home site 110, which includes at least a device 112, a user 113, a test kit 114, one or more samples 115, one or more specimens 116, and a manifest 118; an in-transit phase 140, which includes a tracking number 142 (as well as the sample 115, the one or more specimens 116, and the manifest 118 from the testing/at-home site 110); and a lab 160, which includes a registration 162, a receipt verification 163, a chain of custody 164, digital information 165, a registration 167, a plate sorting 168, storage 170, plate mapping 172, and one or more slots 174 (as well as the sample 115, the one or more specimens 116, and the manifest 118 from the in-transit phase 140).

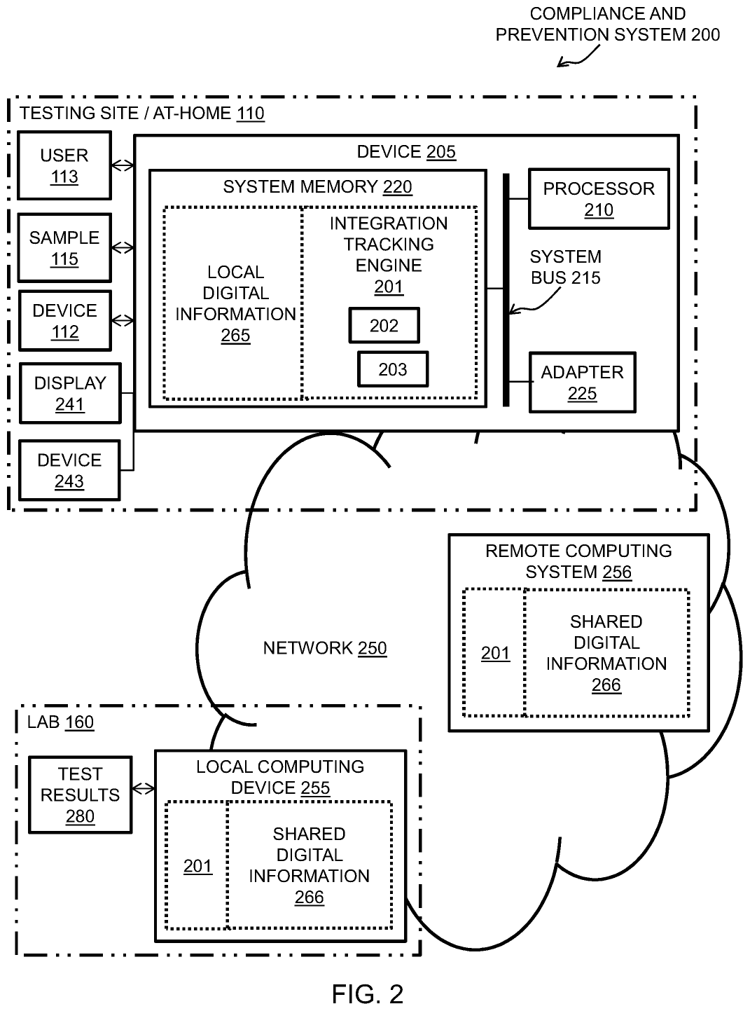

Turning now to FIG. 2, a compliance and prevention system 200 is illustrated according to one or more embodiments. Note that items of FIG. 1 are reused in FIG. 2 for brevity. The compliance and prevention system 200 operates at each stage, location, and operation of the environment 100. For instance, an integration tracking engine 201 provides the necessary software tools, user interfaces, system integrations and the like to ensure integrity and chain of custody 164 of the sample 115 (and specimens 116), as well as any contact tracing or geofencing with respect to the sample 115 (and specimens 116).

Generally, the compliance and prevention system 200 can be representative of the compliance and prevention system and architecture as described herein. In this way, the compliance and prevention system 200 provides to an end-to-end solution that employs the integration tracking engine 201, and other mechanisms and algorithms, to provide users 113 (e.g., patients, customers, companies, and governments) medical data tracking, cross-platform digital management services, and disparate environment integration.

The integration tracking engine 201 can be representative of an operating system for a device 205 for the compliance and prevention system 200 and for the device 112 of the environment 100. According to one or more embodiments, the integration tracking engine 201 can be configured in hardware, software, or a hybrid implementation. The integration tracking engine 201 can be composed of modules that are in operative communication with one another, and to pass information or instructions. The integration tracking engine 201 can further include custom modules to perform application specific processes or derivatives thereof, such that the compliance and prevention system 200 may include additional functionality. For example, according to one or more embodiments, the integration tracking engine 201 may be configured to store information, instructions, commands, or data to be executed or processed by the processor 210 to enable representative operations 202 and 203 (e.g., requisition of the user 113, plate sorting 168 of the sample 115, etc.).

According to one or more embodiments, in view of the representative operations 202 and 203, the integration tracking engine 201 and/or the compliance and prevention system 200 can be implemented with respect to a vaccine process. Note that the vaccine example can be implemented though any UI/GUI of a device (e.g., the device 205) as described herein. Note that, according to one or more embodiments, the vaccine example can include eligibility and registration operations as described herein. For example, the registration operation can include structuring a URL, providing login features, receiving one or more names (e.g., a middle name and mother's maiden name) in a registration form, providing card captures (e.g., taking a picture of an insurance care, social security card, and/or a driver's license). Other data and/or features can relate to user types, testing, vaccinations, etc., along with new patient data, public site data, new company, new vaccination location (e.g., each location may have multiple medical services), schedules, registers via links (e.g., registering for a company can provide access to all locations and medical information), services appointments, site specific link, and user and vaccination type. Other data and/or features can relate to determining which schedule to provide, messaging, auto-scheduling additional appointments, and service/location instructions (e.g., specific pre-arrival instructions).

According to one or more embodiments, in view of the representative operations 202 and 203, the integration tracking engine 201 and/or the compliance and prevention system 200 can implement processes with respect to value stream flows. For instance, the process examples thereof can capture user information, start the plate sorting 168 and mapping 172, and track the samples 115 from point of entry through reporting test, as well as enable creating and printing of one or more labels (e.g., which can be placed on vials and included in a hazard bag as described herein).

Further, embodiments of the compliance and prevention system 200 disclosed may include apparatuses, systems, methods, and/or computer program products at any possible technical detail level of integration. For instance, as shown in FIG. 2, the compliance and prevention system 200 includes the device 205 with one or more central processing units (CPU(s)), which are collectively or generically referred to as a processor 210. The processor 210, also referred to as a processing circuit, is coupled via a system bus 215 to a system memory 220 and various other components. The device 205 may be any computing device, computing apparatus, and/or computing environment, which comprise hardware, software, or a combination thereof (e.g., hardware supporting the integration tracking engine 201). The compliance and prevention system 200 and/or the device 205 may further be adapted or configured to perform as an online platform, a server, an embedded computing system, a personal computer, a console, a personal digital assistant (PDA), a cell phone, a tablet computing device, a quantum computing device, cloud computing device, a mobile device, a smartphone, a fixed mobile device, a smart display, a wearable computer, or the like.

The processor 210 may be any type of general or specific purpose processor, including a central processing unit (CPU), application specific integrated circuit (ASIC), field programmable gate array (FPGA), graphics processing unit (GPU), controller, multi-core processing unit, three dimensional processor, quantum computing device, or any combination thereof. The processor 210 may also have multiple processing cores, and at least some of the cores may be configured to perform specific functions. Multi-parallel processing may also be configured therein. In addition, the processor 210 may be a neuromorphic circuit that includes processing elements that mimic biological neurons.

The system bus 215 (representative of one or more communication mechanism) is configured for communicating information or data to the processor 210, the system memory 220, and various other components, such as the adapter 225.

The system memory 220 is an example of a (non-transitory) computer readable storage medium, where integration tracking engine 201 can be stored as software components, modules, engines, instructions, or the like for execution by the processor 210 to cause the device 205 to operate, such as described herein with reference to any of the Figures. The system memory 220 can include any combination of a read only memory (ROM), a random access memory (RAM), internal or external Flash memory, embedded static-RAM (SRAM), solid-state memory, cache, static storage such as a magnetic or optical disk, or any other types of volatile or non-volatile memory. Non-transitory computer readable storage mediums may be any media that can be accessed by the processor 210 and may include volatile media, non-volatile media, or the like. For example, the ROM is coupled to the system bus 215 and may include a basic input/output system (BIOS), which controls certain basic functions of the device 205, and the RAM is read-write memory coupled to the system bus 215 for use by the processors 210. Non-transitory computer readable storage mediums can include any media that is removable, non-removable, or the like.

The adapter 225 is representative of one or more of the same. Examples of the adapter include, but are not limited to, an input/output (I/O) adapter, a device adapter, and a communications adapter. According to one or more embodiments, the I/O adapter can be configured as a small computer system interface (SCSI), of in view of frequency division multiple access (FDMA) single carrier FDMA (SC-FDMA), time division multiple access (TDMA), code division multiple access (CDMA), orthogonal frequency-division multiplexing (OFDM), orthogonal frequency-division multiple access (OFDMA), global system for mobile (GSM) communications, general packet radio service (GPRS), universal mobile telecommunications system (UMTS), cdma2000, wideband CDMA (W-CDMA), high-speed downlink packet access (HSDPA), high-speed uplink packet access (HSUPA), high-speed packet access (HSPA), long term evolution (LTE), LTE Advanced (LTE-A), 802.11x, Wi-Fi, Zigbee, Ultra-WideBand (UWB), 802.16x, 802.15, home Node-B (HnB), Bluetooth, radio frequency identification (RFID), infrared data association (IrDA), near-field communications (NFC), fifth generation (5G), new radio (NR), or any other wireless or wired device/transceiver for communication. The device adapter interconnects input/output devices to the system bus 215, such as a display 241 and a device 243. The communications adapter interconnects the system bus 215 with a network 250, as described herein, enabling the device 205 to communicate data with other devices. In an embodiment, the adapter 225 may be connected to one or more I/O buses that are connected to the system bus 215 via an intermediate bus bridge. Suitable I/O buses for connecting peripheral devices such as hard disk controllers, network adapters, and graphics adapters typically include common protocols, such as the Peripheral Component Interconnect (PCI).

The display 241 and the device 243 can be representative of one or more devices that are external to the device 205. Examples of the display 241 can include, but are not limited to, a plasma, a liquid crystal display (LCD), a light emitting diode (LED), a field emission display (FED), an organic light emitting diode (OLED) display, a flexible OLED display, a flexible substrate display, a projection display, a 4K display, a high definition (HD) display, a Retina© display, an in-plane switching (IPS) display or the like. The display 241 may be configured as a touch, three dimensional (3D) touch, multi-input touch, or multi-touch display using resistive, capacitive, surface-acoustic wave (SAW) capacitive, infrared, optical imaging, dispersive signal technology, acoustic pulse recognition, frustrated total internal reflection, or the like as understood by one of ordinary skill in the art for input/output (I/O). The display 241, in conjunction with the other elements of FIG. 2, can provide one or more user interfaces (UIs) and/or one or more graphic user interfaces (GUIs) to enable users, software, and the like to interact with the integration tracking engine 201. Examples of the device 243 include, but are not limited to, a scanner, a keyboard, a camera, a speaker, a tablet computer, a mobile device, a computer mouse, a touchpad, a touch screen, a printer, and a keypad. The device 243 may be further coupled to the system bus 215 for input to the device 205.

The network 250 can be a wired network, a wireless network, or include one or more wired and wireless networks that supports communications between all items and elements of the compliance and prevention system 200. According to an embodiment, the network 250 can be an example of a short-range network (e.g., local area network (LAN), or personal area network (PAN)). Information can be sent, via the network 250, between the device 205 and the device 243 using any one of various short-range wireless communication protocols, such as Bluetooth, Wi-Fi, Zigbee, Z-Wave, near field communications (NFC), ultra-band, Zigbee, or infrared (IR). Further, the network 211 is an example of one or more of an Intranet, a local area network (LAN), a wide area network (WAN), a metropolitan area network (MAN), a direct connection or series of connections, a cellular telephone network, or any other network or medium capable of facilitating communication between a local computing device 255 and the remote computing system 256. Information can be sent, via the network 250, using any one of various long-range wireless communication protocols (e.g., TCP/IP, HTTP, 3G, 4G/LTE, or 5G/New Radio). Note that, for the network 250, wired connections can be implemented using Ethernet, Universal Serial Bus (USB), RJ-11 or any other wired connection and wireless connections can be implemented using Wi-Fi, WiMAX, and Bluetooth, infrared, cellular networks, satellite or any other wireless connection methodology.

According to one or more embodiments, the functionality of the device 205 with respect to the integration tracking engine 201 can also be implemented on the local computing device 255 and/or the remote computing system 256, as represented by separate instances of the integration tracking engine 201 therein. In any of the separate instances, the integration tracking engine 201 can perform workflows at test sites, such as registration, patient arrival and check-in, and sample collection. In addition, one or more inputs may be provided to the compliance and prevention system 200 remotely via another computing system (e.g., the local computing device 255 and/or the remote computing system 256) in communication therewith, or the device 205 may operate autonomously.

According to one or more embodiments, the compliance and prevention system 200 at the testing/at-home site 110 that includes the device 205 that used to requisition/register the user 113 and process the sample 115. The device 112 executes an instance of the integration tracking engine 201 (e.g., a client instance, mobile application instance, and/or a kiosk instance). The compliance and prevention system 200 at the lab 160 that includes the local computing device 255. The local computing device 255 executes an instance of the integration tracking engine 201 (e.g., another client instance). The compliance and prevention system 200 can include the network 250 that supports the remote computing system 256. The remote computing system 256 executes an instance of the integration tracking engine 201 (e.g., a backend instance and/or a server instance). In operation, local digital information 265 is collected by the integration tracking engine 201 with respect to the user 113, the sample 115 via the device 112. The local digital information 265 can further be provided to the local computing device 255 and/or the remote computing system 256 and stored therein as shared digital information 266. The local digital information 265 and the shared digital information 266 are examples of the digital information 165 described herein. The local digital information 265 and the shared digital information 266 can be combined and managed through a shared database or other data storage mechanism, as well as encrypted to maintain data integrity and comply with Health Insurance Portability and Accountability Act (HIPAA) requirements. Additionally, as orders are fulfilled with respect to the users 113 and the samples 115, test results 280 are generated and stored across the shared digital information 266.

Returning to FIG. 1, operations of the integration tracking engine 201 within the environment 100, such as a requisition (e.g., the registration of the user 113), order creation (e.g., submitting a test request), and order fulfillment (e.g., generating and reporting the test results 280) are now described.

For requisition, the device 112 is used to register the patient or the customer (i.e., the user 113). By way of example, the device 112 executes an instance or client of the integration tracking engine 201 (as a front end application). Registering, in some cases, includes creating by the integration tracking engine a user profile for the user 113 if that user 113 does not have a user profile. Alternatively, registering can include an identification operation where a user profile corresponding to that user is loaded (e.g., a scanning operation of a bar code or a quick response (QR) code that identifies the user 113 or receiving log-in information). Note that scanning can be performed by any camera or reader integrated with the device 112 and/or in communication with the integration tracking engine 201 and capable of providing scanned information or a picture thereof to the integration tracking engine 201. Then, the integration tracking engine 201 provide a robust data intake when registering the user 113. The robust data intake can include, but is not limited to, providing a user interface with a scalable and configurable set of fields and boxes that engage by the user 113 and receive user information (e.g., as the local digital information 265) with respect to one or more viruses, bacteria, and diseases. The user information can include, but is not limited to, data features with respect to company, user type, sub-user type, policy group, user identifier, first name, last name, email, date of birth, gender, address, phone, race, ethnicity, etc., as well as other demographics tracking, calendars, and reporting data that can be provided directly by the patient. Once registered, an order can be made for the user 113. The order can be a test request for one or more viruses, bacteria, and diseases.

Further, the test kit 114 is used to administer a test for one or more viruses, bacteria, and diseases and/or obtain the sample 115 from the user 113 so the test can be later administered. The test kit 114 can include, but is not limited to, one or more vials, one or more swabs, one or more syringes, one or more bandages, one or more bar codes, one or more labels, paper instructions, and a return envelope. The sample 115 can be mucus, blood, or other bodily fluid obtained by use of the one or more swabs or syringes and/or stored in the one or more vials. In an example, a swab is used to gather mucus from the nose of the user 113 and deposit the mucus into a vial, thereby creating a sample 115. The one or more vials, the one or more swabs, and the one or more syringes can include any commercially available products, as well as customized use specific products.

According to one or more embodiments, the device 112 can identify whether the user 113 is self-serving (i.e., at a domicile or other non-testing site) or a technician is assisting the user 113 (i.e., at a testing site). If the testing/at-home site 110 is the testing site with a technician or a site administrator, the technician can administer the test and/or collect the sample 115. If the testing/at-home site 110 is the domicile or other non-testing site location, the user 113 can self-administer the test and/or collect the sample 115.

According to one or more embodiments, once the sample 115 is collected, the sample 115 can be placed in a vial. Further, one or more specimens 116 can be drawn from the sample 115 and placed in other vials. Each vial can have a bar or QR code thereon. In turn, registration at the testing/at-home site 110 can include a scanning operation of the bar or QR code of each vial to cause the device 112 to associate each vial with the user profile for that user 113 and add/document each vial to the manifest 118, thereby eliminating any paper process. Further, each order associated with each user 113 is also added to or documented on the manifest 118, in correspondence with each sample 115 and each specimen 116. The manifest 118 can be electronically stored with respect to the local digital information 265.

According to one or more embodiments, the manifest 118 is a digital ledger of the integration tracking engine 201 that solves a data management problem by corresponding an order (e.g., a test and a resulting sample 115) and a requisition (e.g., the registration of the user 113), as orders and requisitions are not typically associated/together in conventional mechanisms. In this regard, conventional mechanisms have disparate computing environments at each location that prevents convenient association of information, as well as adds complexity to data privacy in data sharing. In contrast to the conventional mechanisms, the manifest 118 logs information, tracks location statuses, and itemizes progress regarding the sample 115 across the environment 100. The manifest 118 can support multiple samples 115 and multiple specimens 116 obtained within a time range, within a date range, by a same technician, at a particular testing site, or any combination thereof, as well as with respect to a particular shipping parcel. According to one or more embodiments, the manifest 118 is generated with a full listing of included samples 115 and collection date. Note that the manifest 118 enables, at the testing/at-home site 110, the user 113 or technician to initiate the chain of custody 164 for any given sample 115 that is established once the shipping parcel arrives at the lab 160.

The in-transit phase 140, generally, includes operations where the one or more samples 115 and/or one or more specimens 116 are packaged in a shipping parcel, which is assigned a tracking number 142. Further, based to compliance factors, a physical manifest (e.g., printed version of the manifest 118) can be shipped with the samples 115 in shipping parcel and/or stored within a license-plated tracking number. The shipping parcel can include an envelope, a bag, a box, or other container. The integration tracking engine 201 also enables input of the user 113 or the technician who packed the shipping parcel (e.g., the technician scans an identification badge or the user 113 scans their QR code). According to one or more embodiments, the integration tracking engine 201 associates the tracking number 142 and person packing the shipping parcel with the manifest 118 (e.g., the technician who administered the test), which further confirms a next stage in the chain of custody 164. In turn, as a shipping parcel is moved from location to location, the manifest 118 can be updated based on real-time information associated with the tracking number 142. By updating the manifest 118, the status of the one or more samples 115 and/or one or more specimens 116 in the shipping parcel is automatically obtained by the integration tracking engine 201. Note that the manifest 118 can be printed and include a unique bar or QR code thereon itemizing the time and date of the printing, and a printed manifest 118 can be included in the shipping parcel to itemize the one or more samples 115 and/or one or more specimens 116 therein.

The lab 160, generally, is representative of a location where the one or more samples 115 and/or one or more specimens 116 are processed according to the orders of the manifest 118. Process the orders at the lab 160 produces the test results 280. The lab 160 can include the local computing device 255 with the integration tracking engine 201 (as a client application) installed thereon.

A lab technician at the lab 160 performs the registration 162 of the shipping parcel when the shipping parcel arrives at the lab 160. Initially, the lab technician logs into the local computing device 255 with the integration tracking engine 201 installed. Alternatively, the integration tracking engine 201 also enables the technician to scan an identification badge. In this way, the chain of custody 164 is further progressed once the lab technician is identified to the local computing device 255. Further, the lab technician continues the registration 162 by scanning the shipping parcel (e.g., such as the tracking number 142) to perform a receipt verification 163 that triggers population digital information 165 onto the local computing device 255 from the manifest 118. The digital information 165 can include, but is not limited to, user information (including HIPAA data), biometric data, historical data, operational data, tracking data, time stamps, monitoring data, diagnosing data, treatment data, etc. According to one or more embodiments, when the shipping parcel arrives at the lab 160 and is opened, the physical manifest can be scanned (e.g., which causes the integration tracking engine 201 to mark the samples 115 as received on the chain of custody 164).

Additionally, the registration 162 the shipping parcel includes an indication of a next container for all received vials. That is, the integration tracking engine 201 can request input with respect to what type of container, storage, unit, or the like the vial will be placed into. According to one or more embodiments, the storage 170 can be identified (e.g., by scanning a QR or bar code or the plate well or selecting a name of the storage 170 in the integration tracking engine). An example of the storage 170 includes a plate well including one or more slots and a particular length, width, and height. The one or more slots can range in size and/or be uniform, so as to hold the same and/or different types of vials. The length, width, and height of the plate well can be sized to correspond to requirement of one or more testing machines as further described herein. One example of a plate well, though not limited to, is a 96 slot plate well. Thus, by scanning the tracking number 142, loading the manifest 118, and loading the storage 170 at the lab 160, the shipping parcel can be unpacked.

To unpack the shipping parcel, each vial containing each sample 115 or specimen 116 is retried from the shipping parcel and scanned (e.g., checked into the integration tracking engine in a fast efficient way). According to one or more embodiments, the lab technician at the lab 160 performs the registration 167 of each vial of the shipping parcel. The registration includes scanning of a vial as the vial is retrieved from the shipping parcel. This scanning causes the integration tracking engine 201 to automatically provide the plate sorting 168 via a user interface to the lab technician (e.g., once registered each sample 115 can be identified with respect to a plate well). As noted herein, scanning of the shipping parcel, badge, vial, etc. can be performed by any camera or reader device in communication with the integration tracking engine 201 and capable of providing scanned information or a picture thereof to the local computing device 255 of the lab 160.

With plate sorting 168, the integration tracking engine 201 directs the scanned vial to a location within the storage 170 (e.g., guided by the integration tracking engine 201 into a particular slot of the plate well according to the plate mapping 172). Plate mapping 172 enables sample tracking itself within the storage 170 so that at any moment the environment 100 a particular sample is readily locatable, such as an exact slot, a placement time, a lab technician identify, etc. are recorded in the manifest 118. By way of example, the local computing device 255, which executes the integration tracking engine 201 as a client end application, provides the registrations 162 and 167, the plate sorting 168, and the plate mapping 172. All information derived from the registrations 162 and 167, the plate sorting 168, and the plate mapping 172 can be associated with the manifest 118 and included in the shared digital information 266. Further, once the orders are fulfilled at the lab 160, the test results 280 can be generated, associated with the manifest 118 and included in the shared digital information 266, and reported to the user 113. Also, as needed, reporting the test results 280 from the lab 160 to the device 112 or another user device can be performed.

One or more technical effects and benefits include integration of operations across the in-transit phase 140 and the lab 160, with information being supplied, processed, and stored such that the users 113 and the technicians are identified by a log-in, chain of custody is maintained by this identification, and relabeling is eliminated.

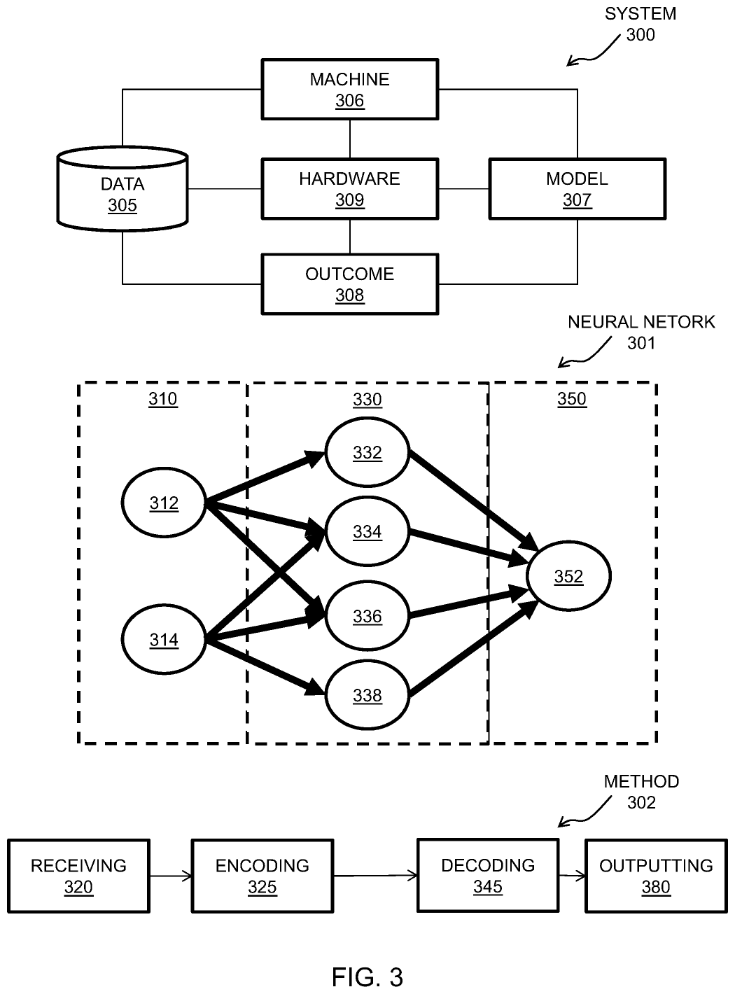

Returning to FIG. 2, additional aspects and capabilities of the integration tracking engine 201 are further described. For instance, in addition to the example operations 202 and 203 of the integration tracking engine 201, the integration tracking engine 201 can includes one or more modules. The modules of the integration tracking engine 201 can be implemented as a hardware circuit comprising custom very large scale integration (VLSI) circuits or gate arrays, off-the-shelf semiconductors such as logic chips, transistors, or other discrete components, in programmable hardware devices (e.g., field programmable gate arrays, programmable array logic, programmable logic devices), graphics processing units, or the like. The modules of the integration tracking engine 201 can be at least partially implemented in software for execution by various types of processors. According to one or more embodiments, an identified unit of executable code may include one or more physical or logical blocks of computer instructions that may, for instance, be organized as an object, procedure, routine, subroutine, or function. Executables of an identified module co-located or stored in different locations such that, when joined logically together, comprise the module. A module of executable code may be a single instruction, one or more data structures, one or more data sets, a plurality of instructions, or the like distributed over several different code segments, among different programs, across several memory devices, or the like. Operational or functional data may be identified and illustrated herein within modules of the integration tracking engine 201 and may be embodied in a suitable form and organized within any suitable type of data structure. An example module of the integration tracking engine 201 includes, but is not limited to, a machine learning and/or an artificial intelligence (ML/AI) module, which is further described with respect to FIG. 3.

FIG. 3 illustrates a graphical depiction of a system 300, an example of a neural network 301, and a block diagram of a network 302 performed in the neural network 301 are shown according to one or more embodiments.

The system 300 includes data 305 (e.g., the digital information 165), a machine 306, a model 307, an outcome 308, and (underlying) hardware 309. For example, the machine 306, the model 307, and the hardware 309 can represent aspects of the integration tracking engine 201, while the hardware 309 can also represent aspects of the compliance and prevention system 200. In general, the ML/AI algorithms of the system 300 (e.g., as implemented by the integration tracking engine 201 and/or the compliance and prevention system 200) operate with respect to the hardware 309, using the data 305, to train the machine 306, build the model 307, and predict the outcomes 308.

For instance, the machine 306 operates as the controller or data collection associated with the hardware 309 and/or is associated therewith. The data 305 can be on-going data or output data associated with the hardware 309. The data 305 can also include currently collected data, historical data, or other data from the hardware 309; can include measurements during a procedure and may be associated with an outcome of the procedure; can include a temperature and/or other sample collected and correlated with the procedure; and can be related to the hardware 309. The data 305 can be divided by the machine 306 into one or more subsets.

Further, the machine 306 trains, such as with respect to the hardware 309. This training can also include an analysis and correlation of the data 305 collected. For example, in the case of testing, the data 305 of sample may be trained to determine if a correlation or link exists between symptoms and diagnosis. In accordance with another embodiment, training the machine 306 can include self-training by the integration tracking engine 201 utilizing the one or more subsets. In this regard, the integration tracking engine 201 learns to detect sample classifications.

Moreover, the model 307 is built on the data 305 associated with the hardware 309. Building the model 307 can include physical hardware or software modeling, algorithmic modeling, and/or the like that seeks to represent the data 305 (or subsets thereof) that has been collected and trained. In some aspects, building of the model 307 is part of self-training operations by the machine 306. The model 307 can be configured to model the operation of hardware 309 and model the data 305 collected from the hardware 309 to predict the outcome 308 achieved by the hardware 309. Predicting the outcomes 308 (of the model 307 associated with the hardware 309) can utilize a trained model 307. Thus, using the outcome 308 that is predicted, the machine 306, the model 307, and the hardware 309 can be configured accordingly.

Thus, for the system 300 to operate with respect to the hardware 309, using the data 305, to train the machine 306, build the model 307, and predict the outcomes 308, the ML/AI algorithms therein can include neural networks (e.g., the neural network 301).

The neural network 301 operates to support implementation of the ML/AI algorithms (e.g., as implemented by the integration tracking engine 201) described herein. The neural network 301 can be implemented in hardware, such as the machine 306 and/or the hardware 309. In general, the neural network 301 is a network or circuit of neurons, or in a modern sense, an artificial neural network (ANN), composed of artificial neurons or nodes or cells. For example, an ANN involves a network of processing elements (artificial neurons) which can exhibit complex global behavior, determined by the connections between the processing elements and element parameters. These connections of the network or circuit of neurons are modeled as weights. A positive weight reflects an excitatory connection, while negative values mean inhibitory connections. Inputs are modified by a weight and summed using a linear combination. An activation function may control the amplitude of the output. For example, an acceptable range of output is usually between 0 and 1, or it could be −1 and 1. In most cases, the ANN is an adaptive system that changes its structure based on external or internal information that flows through the network.

In more practical terms, neural networks are non-linear statistical data modeling or decision-making tools that can be used to model complex relationships between inputs and outputs or to find patterns in data. Thus, ANNs may be used for predictive modeling and adaptive control applications, while being trained via a dataset. Note that self-learning resulting from experience can occur within ANNs, which can derive conclusions from a complex and seemingly unrelated set of information. The utility of ANN models lies in the fact that they can be used to infer a function from observations and also to use it. Unsupervised neural networks can also be used to learn representations of the input that capture the salient characteristics of the input distribution, and more recently, deep learning algorithms, which can implicitly learn the distribution function of the observed data. Learning in the neural network 301 is particularly useful in applications where the complexity of the data (e.g., the digital information 165) or task (e.g., monitoring, diagnosing, and treating any number of various diseases) makes the design of such functions by hand impractical. According to one or more embodiments, the neural network 301 can implement a long short-term memory neural network architecture, a convolutional neural network (CNN) architecture, or other the like. The neural network 301 can be configurable with respect to a number of layers, a number of connections (e.g., encoder/decoder connections), a regularization technique (e.g., dropout), and an optimization feature.

In an example operation, the data 305 is collected from the hardware 309. In the neural network 301, an input layer 310 is represented by a plurality of inputs (e.g., inputs 312 and 314). With respect to block 320 of the network 302, the input layer 310 receives the inputs 312 and 314. The inputs 312 and 314 can include data of the sample 115 (and/or specimen 116).

At block 325 of the network 302, the neural network 301 encodes the inputs 312 and 314 utilizing any portion of the data 305 (e.g., the dataset and predictions produced by the system 300) to produce a latent representation or data coding. The latent representation includes one or more intermediary data representations derived from the plurality of inputs. According to one or more embodiments, the latent representation is generated by an element-wise activation function (e.g., a sigmoid function or a rectified linear unit) of the integration tracking engine 201. The inputs 312 and 314 are provided to a hidden layer 330 depicted as including nodes 332, 334, 336, and 338. The neural network 301 performs the processing via the hidden layer 330 of the nodes 332, 334, 336, and 338 to exhibit complex global behavior, determined by the connections between the processing elements and element parameters. Thus, the transition between layers 310 and 330 can be considered an encoder stage that takes the inputs 312 and 314 and transfers it to a deep neural network (within layer 330) to learn some smaller representation of the input (e.g., a resulting the latent representation).

The deep neural network can be a CNN, a long short-term memory neural network, a fully connected neural network, or combination thereof. The inputs 312 and 314 can be any data received at an intake. This encoding provides a dimensionality reduction of the inputs 312 and 314. Dimensionality reduction is a process of reducing the number of random variables (of the inputs 312 and 314) under consideration by obtaining a set of principal variables. For instance, dimensionality reduction can be a feature extraction that transforms data (e.g., the inputs 312 and 314) from a high-dimensional space (e.g., more than 10 dimensions) to a lower-dimensional space (e.g., 2-3 dimensions). The technical effects and benefits of dimensionality reduction include reducing time and storage space requirements for the data 305, improving visualization of the data 305, and improving parameter interpretation for machine learning. This data transformation can be linear or nonlinear. The operations of receiving (block 320) and encoding (block 325) can be considered a data preparation portion of the multi-step data manipulation by the integration tracking engine 201.

At block 345 of the method 302, the neural network 301 decodes the latent representation. The decoding stage takes the encoder output (e.g., the resulting the latent representation) and attempts to reconstruct some form of the inputs 312 and 314 using another deep neural network. In this regard, the nodes 332, 334, 336, and 338 are combined to produce in the output layer 350 an output 352, as shown in block 380 of the method 302. That is, the output layer 350 reconstructs the inputs 312 and 314 on a reduced dimension but without the signal interferences, signal artifacts, and signal noise. Examples of the output 352 include cleaned data or the like. The technical effects and benefits of the cleaned data include enabling more accurate monitoring, tracking, diagnosis, and treatment any number of various diseases.

According to one or more embodiments, the ML/AI module of the integration tracking engine 201 can, further, keep equipment running at full capacity, develop new processes and implementations, and provide the lab 160 results to review and/or trigger automated decisions, as well as trigger full invoicing to appropriate payee.

Returning to FIG. 2, further aspects and capabilities of the integration tracking engine 201 are further described. For instance, the integration tracking engine 201 can operate to requisition users to generate corresponding user profiles and receive sample data and tracking information for samples corresponding to the users from a test site. The integration tracking engine 201 also generates a manifest that provides automatic sample tracking of the samples as the samples move between the test site and a lab and that provides automatic sample tracking of a processing status of the one or more samples. The integration tracking engine 201 also provides, to the users, test results that are generated by the lab and corresponding to medical orders

According to one or more embodiments, the integration tracking engine 201 can operate to receive a scan of a code on a vial containing a sample. The code at least identifying a user profile associated to the sample and the vial. The integration tracking engine 201 also automatically assigns a position for the vial within a plate map for a plate well, provides the position through a user interface to guide placement of the vial within the plate well, and associates sample data corresponding to the vial and the position within the plate well to a user profile to enable continuous tacking of the sample.

According to one or more embodiments, the integration tracking engine 201 can operate to register vials to record an identity of a user handling the vials and automatically record an identity of a second user in response to receiving corresponding scans of the vials during packaging of the vials into a shipping parcel. Each vial contains a sample collected at a first site and to be tested at a second site. The integration tracking engine 201 also automatically records an identity of a third user in response to receiving corresponding scans of the vials during an unpacking of the vials from the shipping parcel into a plate well and an identity of a fourth user in response to receiving corresponding scans of the vials during testing of the samples in the vials. The recording of the identities of the first and second users establishes and progresses a chain of custody for the vials.

According to one or more embodiments, the integration tracking engine 201 can operate to can operate to detect and scan a bar or quick response (QR) code corresponding to a user profile, load an order for the user profile, generate and present at least one user interface instructing a collecting of a sample within a vial of a test kit and scanning of a bar or QR code of the vial once filled with the sample, and associate the vial with the order, the user profile, and the vial.

According to one or more embodiments, the integration tracking engine 201 can operate to determine whether a user had a positive test with respect to an order, provide one or more notifications to the user indicating that a test was positive when the integration tracking engine 201 determines that the user had the positive test with respect to the order, send an alert to a back-end system to cause a contact tracing operation with respect to the user, and trigger a perimeter management flagging the user as unable to enter one or more facilities.

According to one or more embodiments, the integration tracking engine 201 can operate to detect and scan a bar or quick response (QR) code corresponding to a vial, determine whether o samples corresponding to users are pooled into the vial, associating a pooling order with respect to the vial when the samples are pooled, determines if test results are positive for the vial; and associated a presumptively positive test result with each of the users when test results are positive.

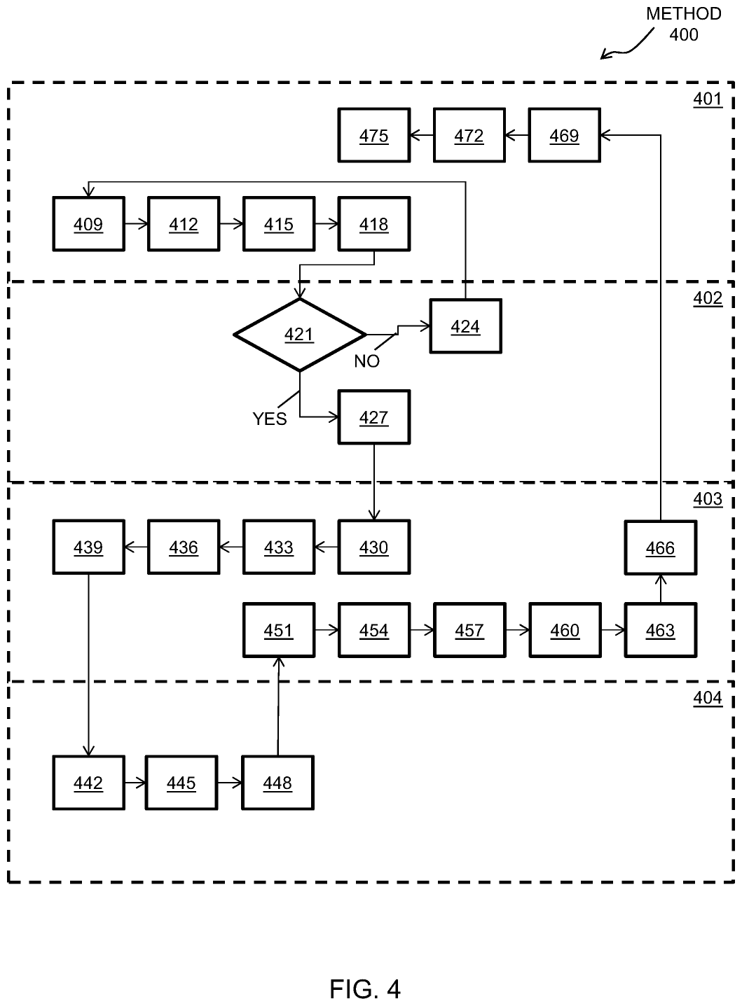

The integration tracking engine 201 can also operate as described with respect to FIGS. 4-9. According to one or more embodiments, FIGS. 4-9 describe example implementations, operations, user interfaces, and outputs of the integration tracking engine 201 and/or the compliance and prevention system 200. Each example can stand alone or be combined with one, more, or all other examples in view of the context of FIGS. 1-3. Note that the example implementations, operations, user interfaces, and outputs of the integration tracking engine 201 and/or the compliance and prevention system 200 are contemplated with respect to one or more UIs and/or GUI that enable interaction between the one or more users 113, one or more disparate environments, one or more different software, and the like.

FIG. 4 depicts a method 400 according to one or more embodiments. The method 400 can be implemented or performed by the integration tracking engine 201. The method 400 relates to an onsite testing process by the integration tracking engine 201 and/or the compliance and prevention system 200, where one or more admins interact with one or more patients (i.e., the user 113). The method 400 addresses a need to address new or emerging health crises by providing a multi-step manipulation of the digital information 165 that enables a robust data intake for patients at testing sites. The method 400 shows operations with respect to a patient portal 401, a check-in portal 402, a registration portal 403, and a testing portal 404, each of which can be considered a user interface of the integration tracking engine 201.

The method begins at block 409, where the integration tracking engine 201 sends a welcome notification, such as an email, a text message, or other communication, to the patient. Generally, a notification can be any communication and/or communication type that alerts the user 113, a technician, and/or other portion of the compliance and prevention system 200. The welcome notification can include a deep link (e.g., uniform resource locator or URL that directs the user 113 to a specific location within a mobile application) and/or a smart link (e.g., uniform resource locator or URL that can be shared, tracked, and customized).

At block 412, the integration tracking engine 201 registers the patient through the patient portal 401. For example, the registration can include, but is not limited to, navigating to a test site marketing website via the welcome notification. The patient submits user information to create a profile and clicks a ‘schedule now’ link. The integration tracking engine 201 redirects the patient to a second website, which presents available testing locations and one or more received selections. At block 415, the integration tracking engine 201, then populates the second website with the calendar showing open days and capacity for a drive timeslot availability. The patient can select date and time and registers or logs-in to the second website. The second website presents payment options with a digital wallet integration, and the patient enters information to remit payment. Once payment is accepted and the appointment is confirmed, the patient receives an email confirmation of the appointment. According to one or more embodiments, the users 113 (e.g., the patients) are registered with all required demographics into the integration tracking engine 201, including compliance and billing requirements with respect to user information (e.g., driver's license, insurance card photos, etc.), and the users 113 schedule testing times. At block 415, the integration tracking engine 201 can detect the arrival of the patient at the test/at-home site 110, for instance by using location services on a mobile device.

At decision block 421, the integration tracking engine 201 presents the check-in portal 402 to confirm whether the patient is registered. If the patient is not registered (as indicated by the NO arrow), the method 400 proceeds to block 424 where an site administrator enters an email address of the patient to prompt a sending of the welcome notification. At block 424, the integration tracking engine 201 can accumulate a number of email addresses to provide a bulk account creation with the compliance and prevention system 200. The method 400 then returns to block 409. If the patient is registered (as indicated by the YES arrow), the method 400 proceeds to block 427. At block 427, the integration tracking engine 201 assigns the patient to a testing queue. In some cases, the site administrator can cause the assignment by providing one or more inputs to the integration tracking engine 201. For example, the patient arrival and check-in can occur at the testing site (e.g., the test/at-home site 110). Further, each testing site can have a physician or other medical personnel that is setup in the integration tracking engine 201. Further, the patient arrival and check-in can include the patient arriving at the testing site in a car at time of appointment and greeted by a technician to check-in. The patient provides the technician with a QR code, which is scanned according. The technician, using the integration tracking engine 201 and the check-in portal 402, verifies patient name and date of birth. The technician, using the integration tracking engine 201 and the check-in portal 402, can also perform a risk assessment regarding whether the user 113 is feeling sick today, has tested positive for a virus or the like in past fourteen (14) days. The technician can complete one or more pre-printed labels (i.e., two matching labels) by typing or writing the name and date of birth of the patient on the one or more labels. The technician scans pre-labeled barcode into the integration tracking engine 201 and the check-in portal 402. Technician applies one or more pre-printed labels to one or more vial (e.g., a first matching label to a vial) and hands labeled vial and additional labels (e.g., a second matching label) to the patient. Patient proceeds to a collection area. The technician completes the patient's order by clicking confirm order on an appointments page of check-in portal 402 and walks through an order confirmation flow, which may include selecting a test type, selecting diagnosis codes, and clicking create order (e.g., which sends completed order, requisition, and patient info for registration). The technician returns to appointments page and approaches next patient.

At blocks 430, 433, 436, and 439, the integration tracking engine 201 presents the registration portal 403 to further confirm whether the patient is registered. Particularly, at block 430, a collector or other site technician scans the QR code of the patient (in some cases this initiates the chain of custody 164). At block 433, the collector scans the first matching label to a vial. At block 436, the collector writes patient identifying information on the vial. At block 439, the collectors provides the patient with the test kit 114 and assigns the patient to an administration queue.

At blocks 442, 445, and 448, the integration tracking engine 201 presents the testing portal 404 for test administration. For example, the sample collection can include a collection completed by sample collector (e.g., a swabber) according to instructions for a particular test. At block 442, the swabber or other site technician confirms a patient name and a date of birth matches the labels and applies the second matching label to a bio-hazard bag or the like. According to one or more embodiments, when the user 113 arrives, the swabber selects a prelabeled test kit, scans the label, and scans the QR code of the user 113 linking the sample 115 to the patient. At block 445, the swabber or other site technician collects the sample 115, inserts the sample 115 into the labeled vial, and inserts the labeled vial into the bio-hazard bag (in some cases continues initiates the chain of custody 164). According to one or more embodiments, if required, the user 113 writes name on the label, and sample is collected and sealed. The bio-hazard bag can contain any number of labeled vial from any number of patients. For instance, at block 448, a predetermined number (e.g., selected from a range of 5-50) or any designated group (e.g., all patients arriving prior to noon or all patients under 18 years of age) is placed in the bio-hazard bag. In this way, samples 115 can be collected on the testing/at-home site in groups (e.g., 20 samples per grouping), which are scanned to create the manifest 118 and “license-plated” to a shipping label.

According to one or more embodiments, the samples 115 can be removed from the shipping parcels and placed in groups, such as groups of 20, 25, 50 or 96. The groups can be placed in clear plastic containers as the samples 115 arrive on sit in the hazard bag. The clear plastic containers can be placed on conveyor belts to move through an assembly line. According to one or more embodiments, plate maps are created, stickered, and reviewed for accuracy (e.g., created by the integration tracking engine 201, linked to a single bar or QR code, and applied to plate wells destined to receive the groups). In an example, over 1,000 plate wells can be processed by the lab 160 a day to test over 100,000 samples 115 per day. Thus, when there are not enough plate wells to support such testing, the samples 115 and corresponding orders are physically held while being processed electronically to advance and simplify collection of user information.

Returning to the registration portal 403 and at block 451, the bio-hazard bag is sealed with the vials therein and further prepared for shipping. For instance, additional identification information can be applied to the bio-hazard bag. At block 454, a courier tracking label is added to the bio-hazard bag. At block 457, all labels are scanned. At block 460, in response to scanning the labels, the manifest 118 is create and/or generated. Further, each label corresponding to each vial on the exterior of the bio-hazard bag can be scanned and added to the manifest 118. With respect to the chain of custody 164, all previous information regarding patient registration, vial labeling, etc. are associated with the manifest 118 so that the chain of custody 164 is maintained. At block 463, the bio-hazard bag is sent to the courier. At block 466, the courier ships the bio-hazard to a destination, such as the lab 160.

Returning to the patient portal 401 and at block 469, the integration tracking engine 201 sends a notification (e.g., a result email) to the patient indicating the test results 280 are available. The notification can include a deep link and/or a smart link. At block 472, the integration tracking engine 201 provides the test results 280 via the patient portal 401, which the patient can access. At block 475, the integration tracking engine 201 provides personalized and customized instructions based on the test results 280 (e.g., instructing to quarantine, seek additional medical attention, etc.) to the patient.

One or more technical effects and benefits of the method 400 include portal integration across each stage of sample collection, with information being supplied, processed, and stored respective to the manifest 118, such that the users 113 and the technicians are identified by a log-in, chain of custody is maintained by this identification, and relabeling is eliminated. Additionally, given current conventional mechanisms, collection and processing of samples is a cumbersome and time consuming process that takes an average of seven days to perform. One or more technical effects and benefits of the method 400 include reducing a time from patient registration to providing test results 280 to less than twenty-four hours.

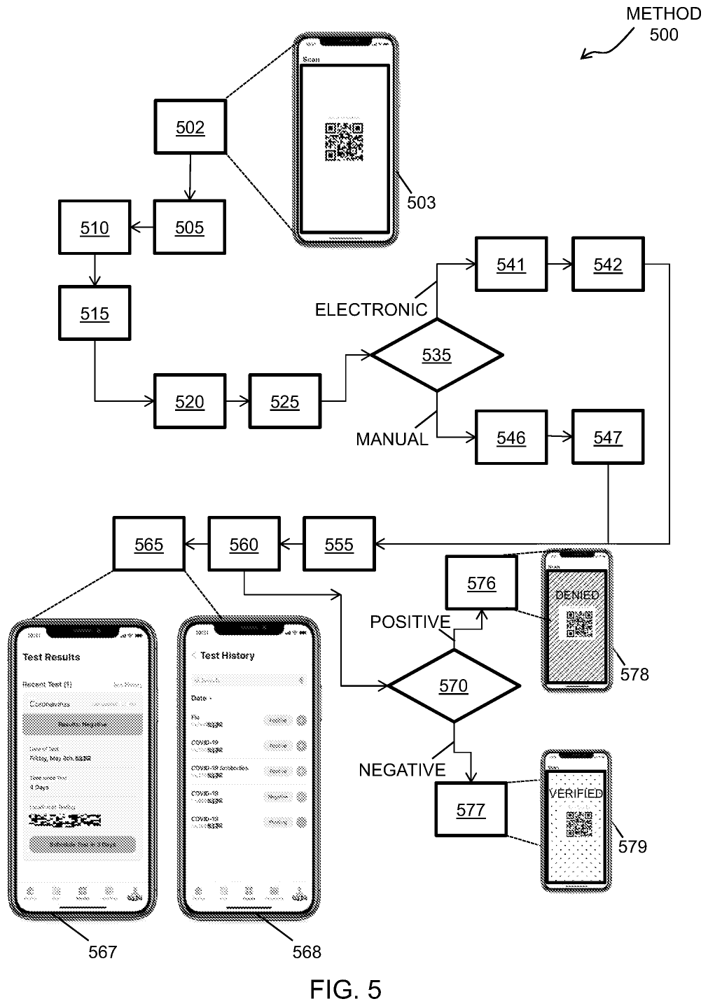

FIG. 5 depicts a method 500 according to one or more embodiments. The method 500 can be implemented or performed by the integration tracking engine 201. The method 500 relates to a sample tracking process (and state processes regarding patient-to-lab states, batch load patient states, and lab process states) by the integration tracking engine 201 and/or the compliance and prevention system 200, where one or more samples 115 are collected from one or more patients (i.e., the users), registered, plate mapped 172, evaluated, and reported. For instance, the method 500 can supplement the method 400 at blocks 466 and 469. The method 500 addresses a need to address new or emerging health crises by providing a multi-step manipulation to track samples 115 with respect to the digital manifest 118 and the digital information 165 to eliminate lost samples, expired samples, lost data, delayed data and/or samples, chain of custody problems, and the like.

The method begins at block 502, where the integration tracking engine 201 registers collects samples. As shown by user interface 503, which is generated by the integration tracking engine 201 in a patient portal 401, an example QR code of a patient can be scanned to initiate the chain of custody 164 (i.e., patient QR scan check-in and registration). At block 505, one or more specimens 116 can be taken from the samples and further distributed into additional vials. Note that each of these addition vials are still associated with the original patient and user information is corresponding loaded into the integration tracking engine 201. At this stage, over 50% of the user information can be provided into the integration tracking engine 201, which is presently unavailable with conventional mechanisms.

At block 510, the samples 115 are retrieved daily by the courier. At block 515, the courier delivers the samples 115. At block 520, the samples 115 are registered by being scanned and cross-checked with the manifest 118. Each scanning loads within a user interface of the integration tracking engine 201 corresponding user information. In some cases, a name and date of birth is confirmed on each vial with respect to the manifest 118. At block 525, once all vial that are received are registered, the integration tracking engine 201 released necessary user information to lab testing systems (e.g., a lab software that runs one or more testing machines). It is noted that without the integration tracking engine 201, conventional mechanisms have no way of efficiently and comprehensively receiving the user information in the lab testing system. The effect of the conventional mechanisms inability to process information is that samples 115 will sit static on a floor or shelf waiting for a manual intake process. By way of example, the manual intake process per vial may average two minutes of data entry, input, and verification. In contrast, the automatic chain of custody 164 and manifest 118 operations by the integration tracking engine 201 decrease the intake process of each vial by over 75%. This improvement enables the samples 115 to be processed the same day and for patients to receive the test results 280 sooner. In this way, the integration tracking engine 201 provides sample tracking by pre-labeling samples 115 imprinted with user information from a quick response (QR) code, scanning sealing samples 115, and manifesting the sealed samples 115 with respect to shipping/tracking details (note that any instance of the integration tracking engine 201 can also perform an early stage prototype of sample tracking).

At decision block 535, after the samples arrive at the lab, the integration tracking engine 201 informs the technician whether the handling of the samples can be electronic or whether the handling requires manual processing. The integration tracking engine 201 makes this determination based on the types of orders associated with the manifest 118. If the handling can be electronic, the method 500 proceeds to block 541. At block 541, the one or more delivered vials are prepared for and processed by one or more testing machines (e.g., the one or more delivered vials are sorted into one or more plate wells according to the plate sorting 168 and the plate mapping 172). At block 542, the one or more plate wells are provided to one or more testing machines. A testing machine, in general, is any laboratory equipment capable of analyzing the samples 115 or specimens 116 in the vials to test for one or more viruses, bacteria, and diseases and/or for conditions indicating the presence of the same. If the handling must be manual, the method 500 proceeds to block 546. At block 546, the one or more delivered vials are prepared for and processed by a lab technician (e.g., the one or more delivered vials are sorted into one or more plate wells according to the plate sorting 168 and the plate mapping 172). At block 547, the one or more plate wells are provided to one or more testing machines or manually tested. According to one or more embodiments with respect to blocks 542 and 547, the one or more testing machines provide DNA extraction from the one or more delivered vials to perform the testing, which results being produce within a time range of 60 minutes 600 minutes (depending on the testing).

At block 555, the integration tracking engine 201 enables additional qualitative diagnostics (e.g., polymerase chain reaction analysis) are performed on the one or more delivered vials and/or the DNA extraction. At block 560, the integration tracking engine 201 enables review of all test results 280, qualitative diagnostics, user information, etc. According to one or more embodiments, a lab technician provides QR scan check-in and register each vial with respect to the corresponding test results 280, as well as pulling up a patient file and order creation page. At block 565, the integration tracking engine 201 provides the test results 280 to the patient. According to one or more embodiments, the integration tracking engine 201 can provide the test results 280 to the patient by push/pull notifications. The patient can also access the patient portal 401 to view the test results and test history, as shown by user interfaces 567 and 568. According to one or more embodiments, the integration tracking engine 201 and/or the compliance and prevention system 200 enables the user 113 to acquire and/or provide details to their user profile, such as practice name, physician name, notional provider identifier number (e.g., NPI #), office phone number, name of office contact, etc. Further, the user interface 567 provides the test results 280 in a visually identifiable way (i.e., color-coded). Other features of the user interfaces 567 and 568 include shipment tracking via one button touch results, text message notifications within two-days, quarantine alerts if the test results 280 are positive, automatic testing alerts in 14 days, and retesting if he test results 280 are negative in a number of days (e.g., three) days or any predetermined sequence. User interface 568 history tracking of individual samples to precisely locate from collection to result (e.g., in real time), as well as a Customized priority level so that a sample is never lost.

Returning to block 560, if the patient indicates to the integration tracking engine 201 that the patient desires to review of the test results 280, the integration tracking engine 201 then determines at decision block 470 whether the results are positive or negative. If the results are positive, the method 500 proceeds to block 576 (as shown by the POSITIVE arrow). If the results are negative, the method 500 proceeds to block 577 (as shown by the NEGATIVE arrow). Accordingly, at block 576, the integration tracking engine 201 generates a user interface 578 that shows in a visually identifiable way (i.e., color-coded as red) the status of the test results 280, as well as any alphanumeric indication as to whether the patient can access a facility, office, stadium, etc. (e.g., the user interface 578 shows a ‘DENIED’ indication). Further, at block 577, the integration tracking engine 201 generates a user interface 579 that shows in a visually identifiable way (i.e., color-coded as green) the status of the test results 280, as well as any alphanumeric indication as to whether the patient can access a facility, office, stadium, etc. (e.g., the user interface 579 shows a ‘VERIFIED’ indication).

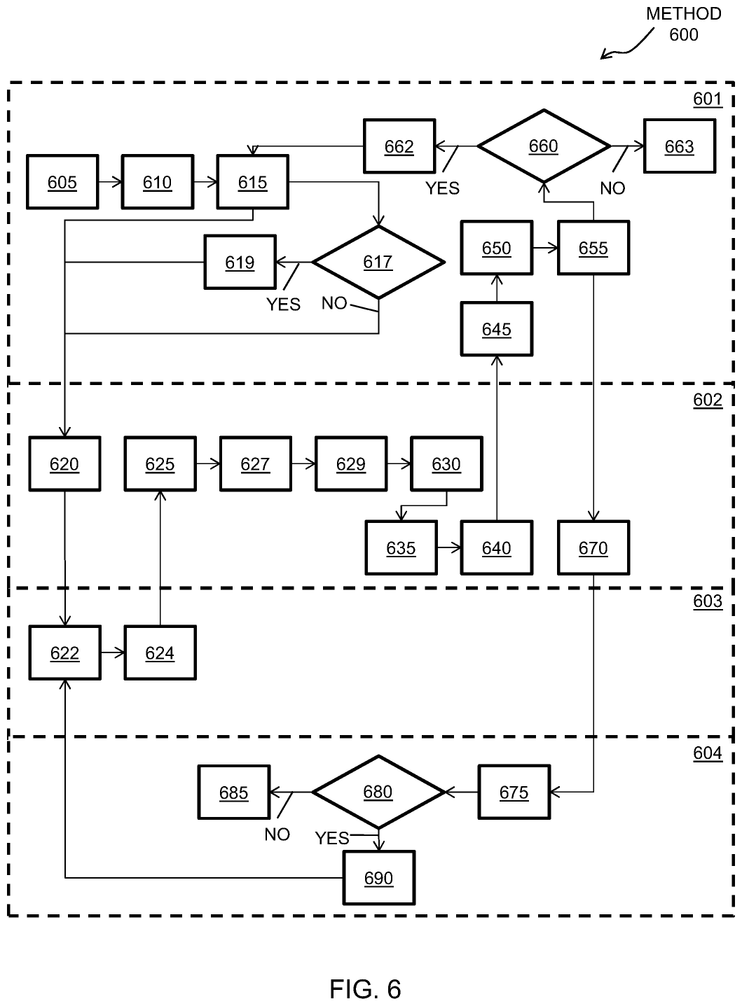

FIG. 6 depicts a method 600 according to one or more embodiments. The method 600 relates to an ordering process by the integration tracking engine 201 and/or the compliance and prevention system 200, where a physician decision power can be exercised in view of one or more evaluated samples 115. The method 600 can be implemented or performed by the integration tracking engine 201. The method 600 addresses a need to address new or emerging health crises by providing one or more portals. The method 600 shows operations with respect to a physician administrative portal 601, a back end software 602, a lab portal 603, and a patient portal 604, each of which can be consider a user interface of the integration tracking engine 201.

At the physician administrative portal 601, the method 600 begins at block 605 where integration tracking engine 201 receives login information from a physician. According to one or more embodiments, at a physician administrative portal 601 (e.g., an example UI/GUI of the integration tracking engine 201), a physician/user can view a patient population, click into a particular patient, and order one or more tests. The integration tracking engine 201 and/or the compliance and prevention system 200 populates one or more panels, provides another collection mechanism, implements a particular collection type, leverages one or more diagnosis codes, and submits one or more orders.

Accordingly and by way of example, at block 610, the integration tracking engine 201 receives examination information regarding the patient including user information regarding an order for testing. The order can be a test for one or more viruses, bacteria, and diseases and/or for conditions indicating the presence of the same. Orders may also include, but are not limited to, review details, review requirements, collected specimen (e.g., name, date of birth, identifying number, collection method, etc.). At block 615, the order is submitted, which directly triggers back end operations by the back end software 602 of the integration tracking engine 201. Additionally, at decision block 617, the integration tracking engine 201 determines whether ‘ask on order entry’ or AOE questions are required based on the order. If AOE questions are not required, the physician administrative portal 601 communicates accordingly to the back end software 602 (e.g., supplements the order with a communication that at the AOE questions are not required). If AOE questions are required, the method 600 proceed to block 619. At block 619, the integration tracking engine 201 presents the physician with the AOE questions, the answer of which are communicated accordingly to the back end software 602. Examples of AOE questions include, but are not limited to, how to collect the sample data and which government agency receive the sample data.

At the back end software 602, the method 600 continues at block 610 where the integration tracking engine 201 receives the order along with any AOE questions. The integration tracking engine 201 associates the order with a ‘patient chart’ (i.e., the user profile for the user 113). One or more technical effects and benefits of the associating of the order with the user profile includes disparate environment integration.

At the lab portal 603 (e.g., an example UI/GUI of the integration tracking engine 201), the method 600 continues at blocks 622 and 624 where one or more samples 115 are collected according to the one or more orders from block 615 and the lab technician completes a requisition/registration of those samples 115. According to one or more embodiments, the integration tracking engine 201 and/or the compliance and prevention system 200 enables via the lab portal 603 logins, patient population view, and an open order view. Any order can be accessed and selected/clicked to open/view the order. The lab technician can add specimen details (e.g., collection date and time, user identifier, tracking info, etc.) and submit the order to the lab 160, create the manifest 118, etc. with respect to block 624, the requisition/registration of the samples 115 by the integration tracking engine 201 is a digital operations that advances the testing process and avoids the use of paper.

The method 600 continues at block 625 the requisition/registration is received by the back end software 602. By way of example, the lab 160 can receive the requisition/registration, such as through a laboratory information management system integrated with the compliance and prevention system 200. At block 627, the integration tracking engine 201 provides tracking of the sample 115 (e.g., once the sample 115 leaves the testing/at-home site 110 and is in the in-transit phase 140. At block 629, the integration tracking engine 201 receives a notification that the sample 115 arrives or is received at the lab 160. At block 630, one or more tests are processed according to the one or more orders associated with the sample at the lab 160.

According to one or more embodiments, the samples 115 are unpacked, scanned, verified, and placed directly into a plate well (e.g., including 96 slots arranged in rows and columns). Each row of the plate well can be identified by one or more distinct characters (e.g., a number), while each column can be identified by one or more distinct characters (e.g., a letter), to form a plate map. For instance, if eight (8) columns can be labeled A through H, then twelve (12) rows can be labeled 1 through 12 to identify each of the 96 slots. As the samples 115 are scanned, a plate map is created by the integration tracking engine 201 and linked to a single bar or QR code, enabling the chain of custody 164 release and receipt to occur at each step with a single scan, as well as being scanned into a computing system of the lab 160, registered with the lab 16, and/or entered into the laboratory information management system. For instance, samples are scanned individually into the plate sorting 168 of the integration tracking engine 201 and assigned a position on, for example, within the 96 slot plate well. The integration tracking engine 201 can populate an image on the lab technician's screen that illustrates and depicts an exact process and placement to ensure a name on the sample 115 matches the imprinted data. The lab technician places each sample in a slot directed by the integration tracking engine 201. According to one or more embodiments, the integration tracking engine 201 guides each vial to a slot by working from A1 through H12. In some cases, the integration tracking engine 201 can leave/maintain three (3) or more consistent slots open in the plate map (e.g., F12, G12, and H12) for control samples. The plate map can be printed or exported as a file. Full plate wells from “CONFIRMED” patients can be released directly into the lab 160. The plate map can be imprinted on 96 tray barcode labels and can be located on the chain of custody 164 based on a plate well identification and precise location in the plate well. The chain of custody 164 and the sample 115 release can be completed by simply scanning the label of the plate well. Each time the plate well is placed in a new location, there is a touch point to scan the label of the plate well to continue marking the chain of custody 164. For example, the samples 115 are scanned before testing, after testing is completed, and into a location in a freezer for storage.

At block 635, the test results 280 are recorded and stored by the integration tracking engine 201. That is, as the lab 160 processed each sample 115 from the plate well, the test results 280 are loaded into the integration tracking engine 201. According to one or more embodiments, all negative results of the test results 280 can be released immediately/automatically, while all positive results of the test results 280 can be reviewed and released by a lab manager and/or physician. At block 640, the integration tracking engine 201 sends one or more notifications to the physician to review the test results 280. Some of the test results 280 can be released immediately/automatically. The release of the test results 280 can be configurable to include safe guards for timely delivery. For instance, based on the type of test and the outcome of the test results 280, the integration tracking engine 201 can be configured to release the test results 280 with respect based on programmed requirements and time.

At the physician administrative portal 601, with respect to block 645, the integration tracking engine 201 provides the test results 280 to the physician to view. At block 650, the integration tracking engine 201 enables the physician to contact the users 113 according to the positive test results. At block 655, the integration tracking engine 201 releases the test results 280 after consulting with the users 113 and/or after a certain time period (e.g., based on based on programmed requirements and time). According to one or more embodiments, the integration tracking engine 201 and/or the compliance and prevention system 200 enables result driven reporting. For instance, the test results 280 can be populated into the physician administrative portal 601, where a physician or practice is alerted to review a test (e.g., order reflex can be required). The user 113 can be contacted, if necessary, and the test results 280 can be released to the patient portal 604. The patient portal 604 can provide an ability to send notes and follow up steps.

At decision block 660, the integration tracking engine 201 can determine if additional testing is required. If additional testing is required, the method 600 proceeds to block 662 (as indicated by the YES arrow) and additional order options are presented by the physician administrative portal 601. The method 600 can further proceed to block 615. If additional testing is NOT required, the method 600 proceeds to block 663 and ends (as indicated by the NO arrow).

Additionally, at the back end software 602 and after block 655, the method 600 continues at block 670 where the integration tracking engine 201 releases the test results 280 to the user profile. One or more technical effects and benefits of the releasing of the test results 280 to the user profile includes disparate environment integration

At the patient portal 604, the method 600 continues at block 675 where the integration tracking engine 201 provides the test results 280 to the patient.

At decision block 680, the integration tracking engine 201 can determine if additional testing is required. If additional testing is not required, the method 600 proceeds to block 685 and ends (as indicated by the NO arrow). According to one or more embodiments, the lab 160, processing time is reduced by the integration tracking engine 201 while the user information and the test results 280 are secured, verified, and transferred electronically (thereby reducing lab redundancies and unnecessary exposure for patient personal identifiers). The integration tracking engine 201 and/or the compliance and prevention system 200 provides patient and client visibility and full process transparency, as well as an ability to customize flows based on client/sample priority, double testing of positive results, and any additional testing preferences. If additional testing is required, the method 600 proceeds to block 690 (as indicated by the YES arrow) and additional sample collection options are presented by the patient portal 604. The method 600 can further proceed to block 622.

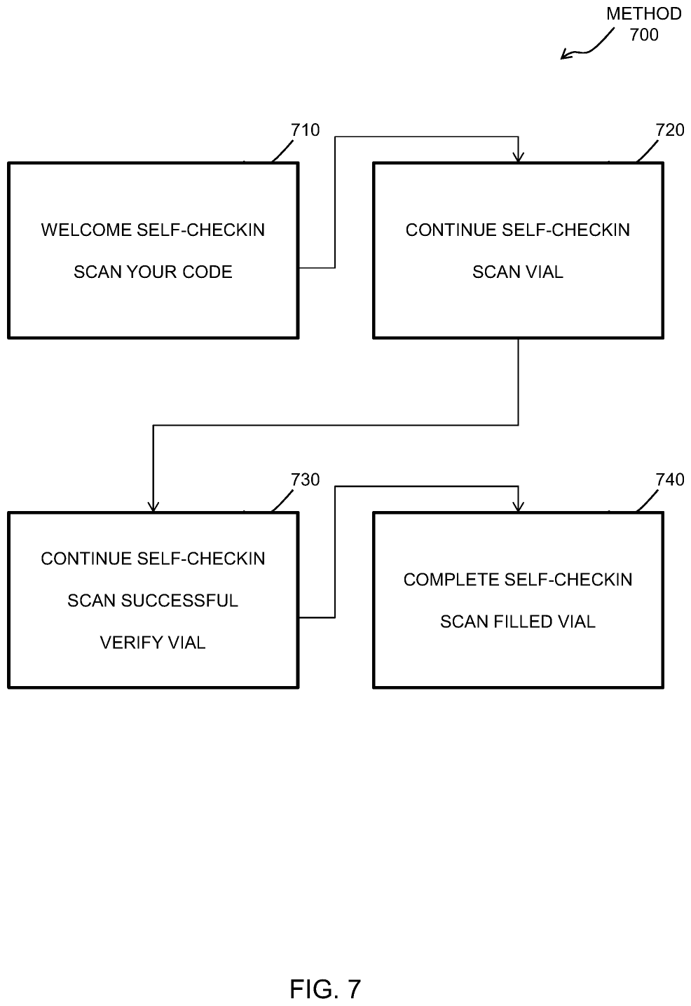

FIG. 7 depicts a method 700 according to one or more embodiments. The method 700 relates to a rapid check-in process by the integration tracking engine 201 with respect to a kiosk instance of the compliance and prevention system 200 (note that the kiosk instance can be implemented as a mobile instance). The kiosk instance can be implemented though any UI/GUI of the integration tracking engine 201 that is presented by the device 205 (e.g., a stand-alone kiosk device). Thus, the kiosk instance can include a set of interfaces or lightweight GUIs that indicate short and well understood commands. In an example, the device 205 is a console with a touch screen and a scanner at the testing site (e.g., testing/at-home site 110), though the kiosk instance can operate independent of human touch so as not to transmit pathogens or diseases between the users 113. The rapid check-in process can be provided through the console. The method 700 addresses a need to address new or emerging health crises by providing smooth and fast mechanisms to check-in patients.

At block 710, the integration tracking engine 201 generates and presents a first interface that greets the user 113. As shown in FIG. 7, the interface can detail “WELCOME SELF-CHECKIN” and “SCAN YOUR CODE”. In response, the user 113 can show the scanner of the console a bar or QR code, whether on paper or on a display. The integration tracking engine 201 detects and reads the bar or QR code, which further triggers the integration tracking engine 201 to locate and load a corresponding user profile. The integration tracking engine 201 loads any orders associated therewith, and creates an order in real-time at the stand-alone kiosk device in view of the corresponding profile. Additionally, the corresponding user profile can include user information, such as protected health information and/or personal identifying information. Note that the detecting and reading of the bar or QR code to identify the user 113 can initiate the chain of custody 164.

At block 720, the integration tracking engine 201 generates and presents a second interface or a scan interface that further instructs the user 113. Generally, the user 113 has already acquired the test kit 114, which includes at least one vial. The vial can include a bar or QR code, as well. In turn, and as shown in FIG. 7, the second interface can detail “CONTINUE SELF-CHECKIN” and “SCAN VIAL”. Additionally, the second interface can specifically direct the user 113 to grab the test kit 114 and take out the vial. In response, the user 113 can show the scanner of the console the bar or QR code of the vial. The integration tracking engine 201 detects and reads this bar or QR code, which further triggers the integration tracking engine 201 to specifically associate that vial with that user 113 and the pending orders that were previously loaded and/or created at block 710.

At block 730, the integration tracking engine 201 generates and presents a third interface or a verify interface that further instructs the user 113. As shown in FIG. 7, the third interface can detail “CONTINUE SELF-CHECKIN”, “SCAN SUCCESSFUL”, and “VERIFY VIAL”. Additionally, the third interface can specifically direct the user 113 to verify that the vial has a number as shown on the third interface, which further confirms that the vial was scanned correctly. The third interface may have an interface button to advance to a fourth interface once the number is verified. Note that the third interface may also have instructions to collect the sample 115.

At block 740, the integration tracking engine 201 generates and presents the fourth interface or a completion interface that further instructs the user 113. As shown in FIG. 7, the fourth interface can detail “COMPLETE SELF-CHECKIN” and “SCAN FILLED VIAL”. Note that the fourth interface may also have instructions to collect the sample 115. For example, the fourth interface may include instructions that indicate the user 113 should write the first and last names on the vial, instructions that direct the user 113 on the method of collecting the sample 115, direct the user 113 to place the filled vial in the biohazard bag, etc. In response, the user 113 can again show the scanner of the console the bar or QR code of the vial. The integration tracking engine 201 again detects and reads this bar or QR code, which further triggers the integration tracking engine 201 to specifically associate the filled vial with that user 113 and the pending order that was previously loaded and/or created at block 710.

According to one or more embodiments, the second, third, and fourth interface of the method 700 can be combined into a single interface so that the user is presented with two screens to check oneself in. In this way, the user 113 can encounter a first interface to scan the bar or QR code and a second interface to scan the filled vial.

One or more technical effects, advantages, or benefits of the integration tracking engine 201 include providing a rapid check-in process that eliminates long wait times, inefficient processing of users 113, and data integrity with respect to data entry and chain of custody problems.

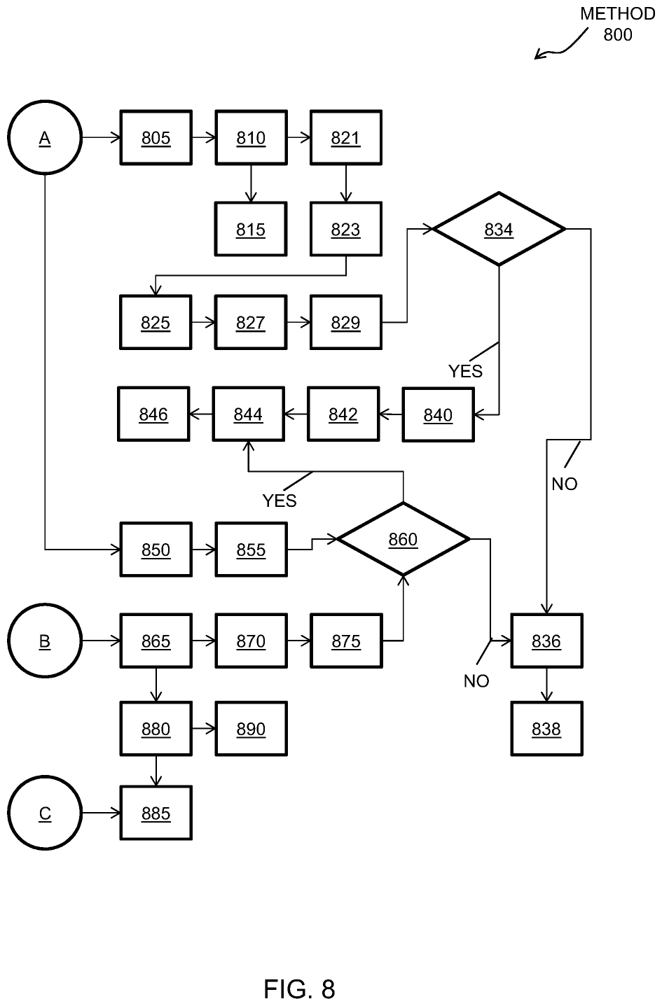

FIG. 8 depicts a method 800 according to one or more embodiments. The method 800 relates to process examples by the integration tracking engine 201 and/or the compliance and prevention system. The method 800 addresses a need to address new or emerging health crises by perimeter management. The method 800 can initiate from a patient A, an employer B, and an administration C (e.g., government office).

The method 800 begins at block 805, where the integration tracking engine 201 is installed on a mobile device for use by the patient A. In turn, at block 810, the integration tracking engine 201 can receive one or more user inputs to enable an registration/requisition of the patient A as described herein. Accordingly, the integration tracking engine 201 can locate and load a corresponding user profile in response to registration/requisition of the patient. At block 815, the integration tracking engine 201 provides one or more features to the patient A, such as proximity searching for a testing location (e.g., utilizing one or more maps and corresponding wait times per location), appointment scheduling (e.g., causing user information to be set to the lab 160), and label and specimen creation. According to one or more embodiments, the integration tracking engine 201 enables capacity scheduling to regulate a flow/movement of patients to each location. In this regard, the integration tracking engine 201 tracks how many users are at and/or scheduled for each testing site and utilizing the ML/AI module to determine how to route the patient A.

At block 821, the integration tracking engine 201 enables the patient A to check-in at a location. According to one or more embodiments, the integration tracking engine 201 can leverage a current location of the mobile device. At block 823, the integration tracking engine 201 can view one or more aspects of a user profile. According to one or more embodiments, the integration tracking engine 201 can enable the patient A to view insurance information, look up insurance status, view order status, view test performed, etc. At block 825, the integration tracking engine 201 can enable the patient A to view completed tests and corresponding test results. At block 827, the integration tracking engine 201 can the patient A to access and view user information in compliance with HIPAA requirements. At block 829, the integration tracking engine 201 can send notifications to the patient, as well as add user information to the test results and test history.

At decision block 834, the integration tracking engine 201 determines if the patient A had a positive test with respect to an order associated with a user profile. If the integration tracking engine 201 determines that the patient A did not have a positive test, then the method 800 proceeds to block 836 (as indicated by the NO arrow). At block 836, the integration tracking engine 201 provides a notifications to the mobile device indicating a positive case was not detected or no contact was made. At block 838, the patient A receives an indication that they can go to work (i.e., that they can enter the perimeter of a facility, building, or the like).

Returning to decision block 834, if the integration tracking engine 201 determines that the patient A did have a positive test, then the method 800 proceeds to block 840 (as indicated by the YES arrow). At block 842, the integration tracking engine 201 provides a notifications to the mobile device indicating the patient A should check the integration tracking engine 201. For example, the integration tracking engine 201 provides one or more notifications to the user indicating that the test was positive (e.g., since the integration tracking engine determined that the user had the positive test with respect to the order). At block 844, the integration tracking engine 201 can send an alert with respect to contact tracing. For instance, the integration tracking engine 201 can send an alert with respect to contact tracing to a back-end system (e.g., of the employer B or the administration C). According to one or more embodiments, integration tracking engine 201 sends one or more alerts to a back-end system to cause a contact tracing operation with respect to the user. The alerts trigger a perimeter management that flags the patient A as unable to enter one or more facilities (e.g., the user interface 578 of FIG. 5). At block 846, the integration tracking engine 201 send quarantine instructions, as well as automatically schedules a re-test. According to one or more embodiments, the integration tracking engine automatically creates an order in real-time and schedules a re-test when the integration tracking engine determines that the user had the positive test with respect to the order to expedite an ability to return to work. Further, the integration tracking engine 201 can provide detailed customized information regarding the virus, the disease, or the bacteria corresponding to the test. The detailed customized information can include, but is not limited to, symptoms, symptoms related to the health patient A, treatment recommendations, and time tables. Additionally, as described herein, the integration tracking engine 201 enables capacity scheduling to regulate a flow/movement of patients to each location with respect to retesting. In this regard, the integration tracking engine 201 tracks how many users are at and/or scheduled for each testing site and utilizing the ML/AI module to determine how to route the patient A.

At block 850, the integration tracking engine 201 enables the patient A to leverage a contact network. In this regard, the integration tracking engine 201 can import contacts from the mobile device, as well as location information, to identify possible real world interactions and exposures. At block 855, the integration tracking engine 201 can integrate with third part contact tracing application programable interfaces (APIs) to determine if contact has been made within the contact network. At decision block 860, the integration tracking engine 201 determines whether there is a positive case and/or a contact has been made. If the integration tracking engine 201 does not detect a positive case or no contact, then the method 800 proceeds to block 836 and 838 (as indicated by the NO arrow). If the integration tracking engine 201 does detect a positive case and a contact, then the method 800 proceeds to block 844 (as indicated by the YES arrow) as described herein.

The method 800 can also begin at block 865, where the integration tracking engine 201 is installed on a company device for use by the employer B. The company device can provide an web interface of the integration tracking engine 201. At block 870, the integration tracking engine 201 enables configuration of testing frequency policies. Testing frequency policies can include, but are not limited to, compliance based configurations in accordance with one or more risk factors. For instance, a high risk environment (e.g., a hospital may have more stringent testing frequency policies than a low risk environment (e.g., a golf club). At block 875, the integration tracking engine 201 can provide daily assessment of each employee (e.g., the patient A). Then, the method 800 proceeds to decision block 860 as described herein.

At block 880, the integration tracking engine 201 enables reporting, such as internal reporting to human resources or reporting out to groups of employees. At block 885, the integration tracking engine 201 enables the incorporation of government data, such as infection rates, so that the further perimeter management can be accomplished. The method 800 can also initiate at block 885, where the integration tracking engine 201 is installed on a government device for use by the administrator C. At block 890, the integration tracking engine 201 enables compliance reporting. According to one or more embodiment, the integration tracking engine 201 can leverage heatmaps, confidence reporting, and worker history and attendance to further assist with perimeter management.

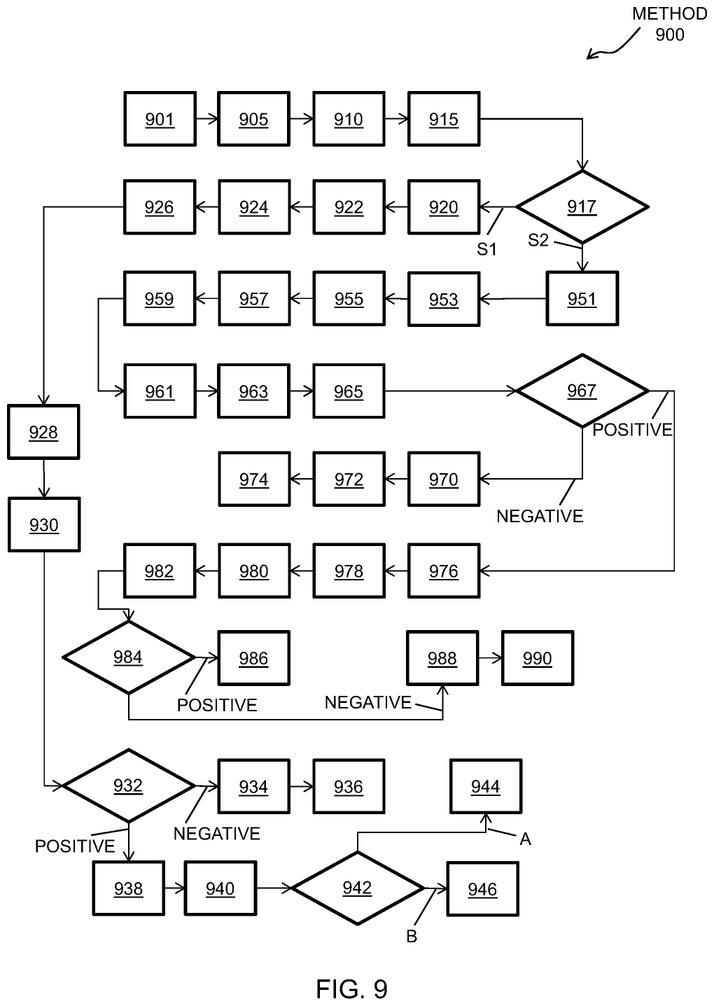

FIG. 9 depicts a method 900 according to one or more embodiments. The method 800 relates to test pooling examples by the integration tracking engine 201 and/or the compliance and prevention system. The method 900 addresses a need to address new or emerging health crises by school or university population (e.g., a test pooling example) by the integration tracking engine 201 and/or the compliance and prevention system 200. Note that test pooling allows for higher efficiency with respect to testing larger groups of a population as the same time.

At block 901, the integration tracking engine 201 registers and creates accounts, such as parent accounts with associated child/student account. The integration tracking engine 201 can include one or more forms, such as minor consent forms. At block 905, the integration tracking engine 201 provides printable QR codes for the child/=student accounts, as well as at least some user information (i.e., full name and date of birth). At block 910, the integration tracking engine 201 identifies an upcoming appointment and waits for one or more patients to arrive. At block 915, the integration tracking engine 201 receives a scan of the QR codes for the child/student accounts. The parent and child/student accounts can be pooled by the integration tracking engine 201. The integration tracking engine 201 also receives a scan of a vial containing samples from each of the child/student accounts corresponding to the QR codes.

At decision block 917, the integration tracking engine 201 determines if one or more samples are pooled within the vial. If one sample is pooled in the vial, then the method 900 proceeds to block 920 (as indicated by the S1 arrow). At block 922, the integration tracking engine 201 determines whether duplicate user data (e.g., same student identification for each child account) is present. At block 924, the integration tracking engine 201 selects one or more patients with respect to the same duplicate user data without having to rescan each vial. At block 926, the integration tracking engine 201 assigns each child/student accounts with its own order. At block 928, the integration tracking engine 201 triggers the lab 160 to receive the vial. At block 930, the integration tracking engine 201 enables the lab 160 to perform the test and record the test results 280.

At decision block 932, the integration tracking engine 201 determines if the test results 280 are positive. If the test results 280 are negative, then the method 900 proceeds to block 934 (as indicated by the NEGATIVE arrow). At block 934, the integration tracking engine 201 provides notifications that the pool is good or valid or safe (i.e., all clear result). At block 936, the integration tracking engine 201 instructs the child/student accounts of the pool to be tested at regular intervals. Regular intervals can be configured according to the need of the school or university. If the test results 280 are positive, then the method 900 proceeds to block 938 (as indicated by the POSITIVE arrow). At block 938, the integration tracking engine 201 provides notifications that all members of the pool are presumptively positive. At block 940, the integration tracking engine 201 provides proper notification and instruction to administrators of the school or university to enable business decisions for the health, safety, and welfare of the pool. The integration tracking engine 201 can also provide quarantine instructions, as well as re-testing instructions, as described herein. The integration tracking engine 201 can also provide further contact tracing, as described herein, with respect to the member of the pool.

At decision block 942, the integration tracking engine 201 determines whether the pool is presumptively positive or if the pool needs to be re-tested. If the integration tracking engine 201 determines that the pool is presumptively positive, then the method 900 proceeds to block 944 (as indicated by the A arrow). If the integration tracking engine 201 determines that the pool needs to be re-tested, then the method 900 proceeds to block 946 (as indicated by the B arrow). At block 944, the integration tracking engine 201 informs by notification or other mechanisms the members of the pool that they are presumptively positive and need to take proper quarantine actions. At block 946, the integration tracking engine 201 informs by notification or other mechanisms the members of the pool need to be re-tested and instructs the child/student accounts of the pool to be tested at regular intervals.

Returning to decision block 917, if two or more samples are pooled, then the method 900 proceeds to block 951 (as indicated by the S2 arrow). At block 951, the integration tracking engine 201 determines whether duplicate user data (e.g., same student identification for each child account) is present. If so, the integration tracking engine 201 will discard any unique identification validation. At block 953, the integration tracking engine 201 selects one or more patients with respect to the same duplicate user data without having to rescan each vial. Further, at block 955, the integration tracking engine 201 assign each member of the pool an order in view of two choices. At block 957, the integration tracking engine 201 presents a first choice of having a pooled sample with a pooled identification. At block 959, the integration tracking engine 201 presents a second choice of a de-convoluted sample with a unique identification. At block 961, the integration tracking engine 201 the manifest 119 identifies the pool and individual samples therein. At block 963, the integration tracking engine 201 enables the lab 160 to receive and register the pooped samples. At block 965, the integration tracking engine 201 enables the lab 160 to perform the test on the pooled sample and record the test results 280.

At decision block 967, the integration tracking engine 201 determines if the test results 280 are positive.

If the test results 280 are negative, then the method 900 proceeds to block 970 (as indicated by the NEGATIVE arrow). At block 970, the integration tracking engine 201 provides notifications that the pool is good or valid or safe (i.e., all clear result). At block 972, the integration tracking engine 201 cancels any follow up orders for testing. At block 974, the integration tracking engine 201 instructs the members of the pool to get tested at regular intervals.

If the test results 280 are positive, then the method 900 proceeds to block 976 (as indicated by the POSITIVE arrow). At block 976, the integration tracking engine 201 locates the physical location of the samples for each member of the pool based on one or more plate well locations as described herein. At block 978, the integration tracking engine 201 registers the samples for each member. At block 980, the integration tracking engine 201 test all samples. At block 982, the integration tracking engine 201 the integration tracking engine 201 provides notifications that all members of the pool are presumptively positive.

At decision block 984, for each sample, the integration tracking engine 201 determines if the test results 280 are positive. If the test results 280 are positive, then the method 900 proceeds to block 986 (as indicated by the POSITIVE arrow). At block 986, the integration tracking engine 201 provides notifications to each member accordingly. If the test results 280 are negative, then the method 900 proceeds to block 988 (as indicated by the NEGATIVE arrow). At block 988, the integration tracking engine 201 overrides the presumptively positive result. At block 990, the integration tracking engine 201 instructs teach member to be tested at regular intervals.

According to one or more embodiments, a computer program product is provided. The computer program product includes processor executable code for an integration tracking engine. The processor executable code is stored on a non-transitory computer readable medium. The processor executable code is executed by at least one processor to cause the integration tracking engine to detect and scan a bar or quick response (QR) code corresponding to a user profile, load an order for the user profile, generate and present at least one user interface instructing a collecting of a sample within a vial of a test kit and scanning of a bar or QR code of the vial once filled with the sample, and associate the vial with the order, the user profile, and the vial.

According to one or more embodiments or any of the computer program product embodiments herein, the integration tracking engine can be implemented in a console comprising the non-transitory computer readable medium and the at least one processor to provide a stand-alone kiosk device.

According to one or more embodiments or any of the computer program product embodiments herein, the integration tracking engine can generate and present a welcome interface to greet a user and request the bar or QR code.

According to one or more embodiments or any of the computer program product embodiments herein, the integration tracking engine can locate and load a corresponding user profile in response to detecting and reading the bar or QR code.

According to one or more embodiments or any of the computer program product embodiments herein, the integration tracking engine can create the order in real-time and in response to detecting and reading the bar or QR code.

According to one or more embodiments or any of the computer program product embodiments herein, the detecting and reading of the bar or QR code can initiate a chain of custody for the vial.

According to one or more embodiments or any of the computer program product embodiments herein, the integration at least one user interface can include a scan interface, a verify interface, and a completion interface.

According to one or more embodiments or any of the computer program product embodiments herein, the integration tracking engine can provide a rapid check-in process with respect to generating and presenting the interface to eliminates wait times and inefficient user processing.

According to one or more embodiments, a method is provided. The method is implemented by an integration tracking engine executing across at least one processor. The method includes detecting and scanning a bar or quick response (QR) code corresponding to a user profile, loading an order for the user profile, generating and presenting at least one user interface instructing a collecting of a sample within a vial of a test kit and scanning of a bar or QR code of the vial once filled with the sample, and associating the vial with the order, the user profile, and the vial.

According to one or more embodiments or any of the method embodiments herein, the integration tracking engine can be implemented in a console comprising non-transitory computer readable medium and the at least one processor to provide a stand-alone kiosk device.

According to one or more embodiments or any of the method embodiments herein, the integration tracking engine can generate and present a welcome interface to greet a user and request the bar or QR code.

According to one or more embodiments or any of the method embodiments herein, the integration tracking engine can locate and load a corresponding user profile in response to detecting and reading the bar or QR code.

According to one or more embodiments or any of the method embodiments herein, the integration tracking engine can create the order in real-time and in response to detecting and reading the bar or QR code.

According to one or more embodiments or any of the method embodiments herein, the detecting and reading of the bar or QR code can initiate a chain of custody for the vial.

According to one or more embodiments or any of the method embodiments herein, the integration at least one user interface can include a scan interface, a verify interface, and a completion interface.

According to one or more embodiments or any of the method embodiments herein, the integration tracking engine can provide a rapid check-in process with respect to generating and presenting the interface to eliminates wait times and inefficient user processing.

According to one or more embodiments, a computer program product is provided. The computer program product includes processor executable code for an integration tracking engine. The processor executable code is stored on a non-transitory computer readable medium. The processor executable code is executed by at least one processor to cause the integration tracking engine to determine whether a user had a positive test with respect to an order, provide one or more notifications to the user indicating that a test was positive when the integration tracking engine determines that the user had the positive test with respect to the order, send an alert to a back-end system to cause a contact tracing operation with respect to the user, and trigger a perimeter management flagging the user as unable to enter one or more facilities.

According to one or more embodiments or any of the computer program product embodiments herein, the integration tracking engine can provide quarantine instructions to the user and detailed customized information regarding a virus, a disease, or a bacteria corresponding to the test.

According to one or more embodiments or any of the computer program product embodiments herein, the integration tracking engine can automatically schedule a re-test when the integration tracking engine determines that the user had the positive test with respect to the order.

According to one or more embodiments or any of the computer program product embodiments herein, the integration tracking engine can automatically create an order in real-time when the integration tracking engine determines that the user had the positive test with respect to the order.

According to one or more embodiments or any of the computer program product embodiments herein, the integration tracking engine can locate and load a corresponding user profile in response registration of the user.

According to one or more embodiments or any of the computer program product embodiments herein, the integration tracking engine can enable capacity scheduling to regulate a movement of users with respect to re-testing when the integration tracking engine determines that the user had the positive test with respect to the order.

According to one or more embodiments or any of the computer program product embodiments herein, the integration tracking engine can track a number of users are at or scheduled for each testing site.

According to one or more embodiments or any of the computer program product embodiments herein, the contact tracing can include utilizing a contact network of the user to determine real world interactions and exposures.

According to one or more embodiments, a method is provided. The method is implemented by an integration tracking engine executing across at least one processor. The method includes determining whether a user had a positive test with respect to an order, providing one or more notifications to the user indicating that a test was positive when the integration tracking engine determines that the user had the positive test with respect to the order, sending an alert to a back-end system to cause a contact tracing operation with respect to the user, and triggering a perimeter management flagging the user as unable to enter one or more facilities.

According to one or more embodiments or any of the method embodiments herein, the integration tracking engine can provide quarantine instructions to the user and detailed customized information regarding a virus, a disease, or a bacteria corresponding to the test.

According to one or more embodiments or any of the method embodiments herein, the integration tracking engine can automatically schedule a re-test when the integration tracking engine determines that the user had the positive test with respect to the order.

According to one or more embodiments or any of the method embodiments herein, the integration tracking engine can automatically create an order in real-time when the integration tracking engine determines that the user had the positive test with respect to the order.

According to one or more embodiments or any of the method embodiments herein, the integration tracking engine can locate and load a corresponding user profile in response registration of the user.

According to one or more embodiments or any of the method embodiments herein, the integration tracking engine can enable capacity scheduling to regulate a movement of users with respect to re-testing when the integration tracking engine determines that the user had the positive test with respect to the order.

According to one or more embodiments or any of the method embodiments herein, the integration tracking engine can track a number of users are at or scheduled for each testing site.

According to one or more embodiments or any of the method embodiments herein, the contact tracing can include utilizing a contact network of the user to determine real world interactions and exposures.

According to one or more embodiments, a computer program product is provided. The computer program product includes processor executable code for an integration tracking engine. The processor executable code is stored on a non-transitory computer readable medium. The processor executable code is executed by at least one processor to cause the integration tracking engine to perform detecting and scanning a bar or quick response (QR) code corresponding to a vial, determining whether one or more samples corresponding to one or more users are pooled into the vial, associating a pooling order with respect to the vial when the one or more samples are pooled, determines if test results are positive for the vial, and associated a presumptively positive test result with each of the one or more users when test results are positive.

According to one or more embodiments or any of the computer program product embodiments herein, the integration tracking engine can register and create accounts for one or more users.

According to one or more embodiments or any of the computer program product embodiments herein, the integration tracking engine can provide one or more notifications to each of the one or more users when test results are positive.

According to one or more embodiments or any of the computer program product embodiments herein, the integration tracking engine can provide one or more notifications and instruction to administrators when test results are positive.

According to one or more embodiments or any of the computer program product embodiments herein, the integration tracking engine can provide quarantine and re-testing instructions to each of the one or more users when test results are positive.

According to one or more embodiments or any of the computer program product embodiments herein, the =integration tracking engine can assign each of the one or more users an order in view of at least two choices comprising having a pooled sample with a pooled identification and a de-convoluted sample with a unique identification.

According to one or more embodiments or any of the computer program product embodiments herein, the integration tracking engine can associate an all clear result with each of the one or more users when test results are negative.

According to one or more embodiments or any of the computer program product embodiments herein, the integration tracking engine can instruct each of the one or more users to be tested at regular intervals based on the all clear result.

According to one or more embodiments, a method is provided. The method is implemented by an integration tracking engine executing across at least one processor. The method includes detecting and scanning a bar or quick response (QR) code corresponding to a vial, determining whether one or more samples corresponding to one or more users are pooled into the vial, associating a pooling order with respect to the vial when the one or more samples are pooled, determines if test results are positive for the vial, and associated a presumptively positive test result with each of the one or more users when test results are positive.

According to one or more embodiments or any of the method embodiments herein, the integration tracking engine can register and create accounts for one or more users.

According to one or more embodiments or any of the method embodiments herein, the integration tracking engine can provide one or more notifications to each of the one or more users when test results are positive.

According to one or more embodiments or any of the method embodiments herein, the integration tracking engine can provide one or more notifications and instruction to administrators when test results are positive.

According to one or more embodiments or any of the method embodiments herein, the integration tracking engine can provide quarantine and re-testing instructions to each of the one or more users when test results are positive.

According to one or more embodiments or any of the method embodiments herein, the integration tracking engine can assign each of the one or more users an order in view of at least two choices comprising having a pooled sample with a pooled identification and a de-convoluted sample with a unique identification.

According to one or more embodiments or any of the method embodiments herein, the integration tracking engine can associate an all clear result with each of the one or more users when test results are negative.

According to one or more embodiments or any of the method embodiments herein, the integration tracking engine can instruct each of the one or more users to be tested at regular intervals based on the all clear result.

The flowchart and block diagrams in the drawings illustrate the architecture, functionality, and operation of possible implementations of apparatuses, systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the flowchart and block diagrams in the drawings. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although features and elements are described above in particular combinations, one of ordinary skill in the art will appreciate that each feature or element can be used alone or in any combination with the other features and elements. For instance, for any of the methods and processes described herein, the steps recited may be performed out of sequence in any order and sub-steps not explicitly described or shown may be performed. When using referring to “A or B”, it may include A, B, or A and B, which may be extended similarly to longer lists. When using the notation X/Y it may include X or Y. Alternatively, when using the notation X/Y it may include X and Y. X/Y notation may be extended similarly to longer lists with the same explained logic. In addition, “coupled” or “operatively coupled” may mean that objects are linked but may have zero or more intermediate objects between the linked objects. Also, any combination of the disclosed features/elements may be used in one or more embodiments.

In addition, the methods and processes described herein may be implemented in a computer program, software, and/or firmware (e.g., a computer program product) incorporated in a computer-readable medium for execution by a computer or processor. That is, the computer program product may include a computer readable storage medium (or media) having computer readable program instructions thereon for causing a controller, processor, or the like to carry out aspects of the present invention.

The computer readable storage medium can be a tangible device that can retain and store computer readable program instructions. The computer readable storage medium may be, for example, but is not limited to, an electronic storage device, a magnetic storage device, an optical storage device, an electromagnetic storage device, a semiconductor storage device, or any suitable combination of the foregoing. The computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Examples of computer-readable storage media include, but are not limited to, a register, cache memory, semiconductor memory devices, magnetic media such as internal hard disks and removable disks, magneto-optical media, optical media such as compact disks (CD) and digital versatile disks (DVDs), a random access memory (RAM), a read-only memory (ROM), an erasable programmable read-only memory (EPROM or Flash memory), a static random access memory (SRAM), and a memory stick.

The computer readable program instructions described herein can be communicated and/or downloaded to respective controllers, processors, or the like from an apparatus, device, computer, or external storage via a connection, for example, network communications. Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used herein, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A computer program product comprising processor executable code for an integration tracking engine, the processor executable code being stored on a non-transitory computer readable medium, the processor executable code being executed by at least one processor to cause the integration tracking engine to perform:
detecting and scanning a bar or quick response (QR) code corresponding to a vial;
determining whether one or more samples corresponding to one or more users are pooled into the vial;
associating a pooling order with respect to the vial when the one or more samples are pooled;
determines if test results are positive for the vial; and
associated a presumptively positive test result with each of the one or more users when test results are positive.

2. The computer program product of claim 1, wherein the integration tracking engine registers and creates accounts for one or more users.

3. The computer program product of claim 1, wherein the integration tracking engine provides one or more notifications to each of the one or more users when test results are positive.

4. The computer program product of claim 1, wherein the integration tracking engine provides one or more notifications and instruction to administrators when test results are positive.

5. The computer program product of claim 1, wherein the integration tracking engine provides quarantine and re-testing instructions to each of the one or more users when test results are positive.

6. The computer program product of claim 1, wherein the integration tracking engine assigns each of the one or more users an order in view of at least two choices comprising having a pooled sample with a pooled identification and a de-convoluted sample with a unique identification.

7. The computer program product of claim 1, wherein the integration tracking engine associates an all clear result with each of the one or more users when test results are negative.

8. The computer program product of claim 7, wherein the integration tracking engine instructs each of the one or more users to be tested at regular intervals based on the all clear result.

9. A method implemented by an integration tracking engine executing across at least one processor, the method comprising:
detecting and scanning a bar or quick response (QR) code corresponding to a vial;
determining whether one or more samples corresponding to one or more users are pooled into the vial;
associating a pooling order with respect to the vial when the one or more samples are pooled;
determines if test results are positive for the vial; and
associated a presumptively positive test result with each of the one or more users when test results are positive.

10. The method of claim 9, wherein the integration tracking engine registers and creates accounts for one or more users.

11. The method of claim 9, wherein the integration tracking engine provides one or more notifications to each of the one or more users when test results are positive.

12. The method of claim 9, wherein the integration tracking engine provides one or more notifications and instruction to administrators when test results are positive.

13. The method of claim 9, wherein the integration tracking engine provides quarantine and re-testing instructions to each of the one or more users when test results are positive.

14. The method of claim 9, wherein the integration tracking engine assigns each of the one or more users an order in view of at least two choices comprising having a pooled sample with a pooled identification and a de-convoluted sample with a unique identification.

15. The method of claim 9, wherein the integration tracking engine associates an all clear result with each of the one or more users when test results are negative.

16. The method of claim 15, wherein the integration tracking engine instructs each of the one or more users to be tested at regular intervals based on the all clear result.
